(19) United States
(12) Patent Application Publication
(10) Pub. No.: US 2025/0382599 A1
(43) Pub. Date: Dec. 18, 2025

(54) VIRAL LOAD-DEPENDENT CRISPR/CAS13-SYSTEM

(71) Applicant: Helmholtz Zentrum München Deutsches Forschungszentrum für Gesundheit und Umwelt (GmbH), Neuherberg (DE)

(72) Inventors: Wolfgang Wurst, Muenchen (DE); Christoph Gruber, Freising (DE); Lea Krautner, Muenchen (DE); Florian Giesert, Muenchen (DE)

(21) Appl. No.: 18/879,172

(22) PCT Filed: Jun. 27, 2023
(86) PCT No.: PCT/EP2023/067472

§ 371 Date: Dec. 26, 2024

(30) Foreign Application Priority Data
Jun. 27, 2022 (EP) 22181376.9

(51) Int. Cl. C12N 9/22; C12N 15/11

(52) U.S. Cl. CPC C12N 9/226; C12N 15/11; C07K 2319/09; C07K 2319/095; C12N 2310/20

(57) ABSTRACT

The present invention relates to a novel CRISPR system comprising i) at least one nucleotide sequence encoding at least one Cas13 protein; and ii) at least one gRNA or at least one nucleotide sequence encoding said at least one gRNA capable of hybridizing with one or more viral target RNA molecules, wherein said system comprises a viral 5′ UTR or a nucleotide sequence encoding said 5′ UTR and/or a viral 3′ UTR or a nucleotide sequence encoding said viral 3′ UTR, wherein a viral replicase recognition sequence is comprised in at least any one of said 5′ UTR or in the nucleotide sequence encoding said 5′ UTR, or said 3′ UTR or in the nucleotide sequence encoding said 3′ UTR, and wherein said system does not comprise a nucleotide sequence encoding a viral replicase and wherein said viral replicase recognition sequence is from the same RNA virus as the one or more viral target RNA molecules. The present invention also relates to a delivery system comprising the novel system and a composition comprising the novel system or the delivery system. The present invention further relates to the medical use of the novel system or in particular to the system for use in a method of preventing or treating a viral disease in a subject. Additionally, the present invention also relates to a kit comprising the novel system and to a method of producing the novel system.

### CROSS-REFERENCE TO RELATED APPLICATIONS

The present application claims the benefit of priority of EP patent application No. 22181376.9 filed 27 Jun. 2022, the content of which is hereby incorporated by reference in its entirety for all purposes.

### FIELD OF THE INVENTION

The present invention relates to a novel CRISPR system comprising i) at least one nucleotide sequence encoding at least one Cas 13 protein; and ii) at least one gRNA or at least one nucleotide sequence encoding said at least one gRNA capable of hybridizing with one or more viral target RNA molecules, wherein said system comprises a viral 5′ UTR or a nucleotide sequence encoding said 5′ UTR and/or a viral 3′ UTR or a nucleotide sequence encoding said viral 3′ UTR, wherein a viral replicase recognition sequence is comprised in at least any one of said 5′ UTR or in the nucleotide sequence encoding said 5′ UTR, or said 3′ UTR or in the nucleotide sequence encoding said 3′ UTR, and wherein said system does not comprise a nucleotide sequence encoding a viral replicase and wherein said viral replicase recognition sequence is from the same RNA virus as the one or more viral target RNA molecules. The present invention also relates to a delivery system comprising the novel system and a composition comprising the novel system or the delivery system. The present invention further relates to the medical use of the novel system or in particular to the system for use in a method of preventing or treating a viral disease in a subject. Additionally, the present invention also relates to a kit comprising the novel system and to a method of producing the novel system.

### BACKGROUND OF THE INVENTION

From a clinical point of view, the problem with the rapid spread of virus or bacterial strains in general is that the development of a drug takes too much time to be able to develop a therapeutic agent in a reasonable time. Especially with regard to the novel coronavirus SARS-CoV-2 which is a (+)-RNA virus of the Coronaviridae family and which as of early October 2020 has caused over 1.000.000 deaths worldwide, drug development is a critical issue. RNA viruses are also responsible for the other two epi- and pandemics of the recent past (SARS-CoV-1 and MERS-CoV). These two epidemics have in common with COVID-19 to have a high virulence combined with an efficient route of spreading via droplet infections (P. Anfinrud et al. (2020), N Engl J Med 382, 2061-2063; J. Chen (2020) Microbes Infect 22, 69-71).

For this reason, the only means available is the “repurposing” of drugs that have already been approved. For example, Remdesivir, which has originally been developed as a drug for Ebola, is currently being discussed as a therapy for COVID-19 (J. Grein et al. (2020), N Engl J Med 382, 2327-2336). However, since antibodies and small molecule-based therapies such as Remdesivir use the tertiary structure of a protein as the target structure, it is often not possible to apply an approved inhibitor to a new virus or bacteria strain and in addition, viral or bacterial mutations can also change the binding properties of a therapeutic agent.

The prokaryotic immune system CRISPR/Cas acts differently than the immune system of higher eukaryotes. Instead of binding to protein antigens, the CRISPR/Cas systems directly recognize the genetic information of a phage at the ribonucleic acid level. By simply expressing a guide RNA (gRNA) that is complementary to the phage genome, an effector nuclease is directed to the genome of the phage and cutting of the genome is induced (F. Hille et al. (2018), Cell 172, 1239-1259). CRISPR/Cas systems are divided into two classes with six types (K. S. Makarova et al. (2020), Nat Rev Microbiol 18, 67-83). In addition to Cas9, as a programmable DNAse, Cas13 was recently discovered. Unlike Cas9, Cas13 does not cut the DNA but rather the RNA of a phage that attacks the prokaryotic host (O. O. Abudayyeh et al. (2016), Science 353). The nuclease of the Cas13 effector is a HEPN (higher eukaryotes and prokaryotes nucleotide-binding) domain that is split and therefore inactive within the protein. As soon as a target RNA is bound, there is a change in the tertiary structure of the protein, whereby the separated nuclease domain is brought into proximity and activated (O. O. Abudayyeh et al. (2016)).

In the prior art different approaches with Cas13 have already been demonstrated for some model viruses such as influenza A and in an artificial experimental system transfecting synthetic SARS-Cov-2 sequences into human cells expressing already Cas13 (C. A. Freije et al. (2019), Mol Cell 76, 826-837, Abbott et al. (2020), Cell 181, 865-876). Despite the current success of developing vaccines against a human-pathogenic virus, there remains a need to provide alternative or improved systems, compositions and methods for treatment and/or prevention of diseases caused by human-pathogenic viruses. The technical problem underlying the present application is thus to comply with this need.

The technical problem is solved by providing the embodiments reflected in the claims, described in the description and illustrated in the examples and figures that follow.

### SUMMARY OF THE INVENTION

The novel CRISPR/Cas13 system according to the present invention represents a new antiviral therapy approach for the treatment of RNA-based viral diseases. It utilizes the RNase activity of the so-called Cas13 protein. In particular, a viral mRNA that has been introduced into or replicated in the cell is specifically degraded by the novel clustered, regularly interspaced, short palindromic repeats (CRISPR) system comprising at least one nucleotide sequence encoding at least one CRISPR-associated protein 13 (Cas13) and at least one guide RNA (gRNA) or at least one nucleotide sequence encoding said at least one gRNA. This particular and novel system also comprises at least any one of a viral 5′ untranslated region (UTR) or a nucleotide sequence encoding said viral 5′ UTR or a viral 3′ UTR or a nucleotide sequence encoding said viral 3′ UTR, wherein a viral replicase recognition sequence as viral replication element is comprised/located in at least any one of said viral 5′ UTR or in the nucleotide sequence encoding said viral 5′ UTR, or said viral 3′ UTR or in the nucleotide sequence encoding said viral 3′ UTR. Such system can be applied on the DNA or RNA level. Further, said novel system does not comprise a nucleotide sequence encoding a viral replicase. In an application of said system against for example SARS-CoV-2, the inventors have demonstrated an 85% reduction in viral load in cell culture experiments (see FIG. 4). An improvement of this antiviral approach based on the novel system according to the present invention thus increases the therapeutic efficiency.

In sum, the genetic information (on the DNA or RNA level) of the CRISPR/Cas13 system elements of said system being flanked by at least one viral 5′ UTR or said at least one nucleotide sequence encoding said at least one viral 5′ UTR and at least one viral 3′ UTR or said at least one nucleotide sequence encoding said at least one viral 3′ UTR, in which—if both or more viral UTRs are applied within said system—a viral replicase recognition sequence is located either within the viral 5′ UTR or the nucleotide sequence encoding said 5′ UTR, or within the viral 3′ UTR or the nucleotide sequence encoding said 3′ UTR, or in both, refers to the main characteristic of said novel system (see FIGS. 1 and 5). The same applies mutatis mutandis to the system as defined above, when either the viral 5′ UTR or the nucleotide sequence encoding said viral 5′ UTR or the viral 3′ UTR or the nucleotide sequence encoding said viral 3′ UTR is applied within said system, meaning that the viral replicase recognition sequence is then either located within the viral 5′ UTR or the nucleotide sequence encoding said viral 5′ UTR, or within the viral 3′ UTR or the nucleotide sequence encoding said viral 3′ UTR. Thus, the presence of proteins of the virus itself, particularly the viral replicase enzyme, is used to replicate the initially applied Cas13 mRNA using the protein synthesis machinery of the virus to be treated respectively, thereby increasing the intracellular concentration of the therapeutically effective Cas13 protein in infected cells.

The inventors have shown by model calculations on the development of the viral load in an application of the system as defined herein that the amount of Cas13d mRNA is directly correlated to the viral load, signifying a faster and stronger increase in Cas13 observed at a high viral load than at a low viral load due to co-replication and co-distribution with a wave-like dependence of the concentration of viral and system components over time. Thus, the application of the CRISPR/Cas 13 system of the invention which is co-replicated by the virus having infected the cells of the subject lead to a complete elimination of the virus as predicted by a mathematical model (see FIG. 2). In sum, the system of the invention as defined herein is able to self-amplify in dependency of the viral load and is thus considered an antiviral agent. For in vivo application, the amplification mechanism according to the invention allows for a high quantity correlation of 1:1 predicted as necessary to achieve therapeutic efficiency and quickly fulfill the required efficacy, especially in severe disease progressions, to be achieved with a much lower dose of antiviral initially applied to the organism with a subsequent dose adjustment taking place directly in vivo. Said co-dependence of protein levels as an essential prerequisite for successful therapeutic use against viral infections with high viral loads like SARS-Cov-2 is shown in SARS-Cov-2 transfected Vero 6 cells via co-translation of fluorescent reporter constructs from the same mRNA strand as the Cas 13 protein (see FIGS. 3 and 4).

The novel system as defined above comprises different system variants as it is described by FIGS. 5, 6, 7 and 8 and which reduce, when each is applied, the viral load in cell culture experiments, thus increasing the therapeutic efficiency when used in therapy, in particular when applied to prevent or treat a viral disease in a subject as defined herein. All of the different system variants comprise at least one viral UTR (at least one viral 5′ and/or at least one 3′ UTR) or—at the DNA level—at least one nucleotide sequence encoding said at least one viral UTR, wherein the viral replicase recognition sequence as viral replication element, is comprised in at least any one of said viral 5′ UTR or in the nucleotide sequence encoding said 5′ UTR, or said viral 3′ UTR or in the nucleotide sequence encoding said 3′ UTR. In addition, all of the different system variants do not comprise a nucleotide sequence encoding a viral replicase. Again, each system variant is able to self-amplify dependent on the viral load and are thus considered as antiviral agents.

Accordingly, in a first aspect, the present invention relates to a CRISPR system comprising i) at least one nucleotide sequence encoding at least one Cas13 protein; and ii) at least one gRNA or at least one nucleotide sequence encoding said at least one gRNA capable of hybridizing with one or more viral target RNA molecules, wherein said system comprises a viral 5′ UTR or a nucleotide sequence encoding said 5′ UTR and/or a viral 3′ UTR or a nucleotide sequence encoding said viral 3′ UTR, wherein a viral replicase recognition sequence is comprised in at least any one of said 5′ UTR or in the nucleotide sequence encoding said 5′ UTR, or said 3′ UTR or in the nucleotide sequence encoding said 3′ UTR, and wherein said system does not comprise a nucleotide sequence encoding a viral replicase and wherein said viral replicase recognition sequence is from the same RNA virus as the one or more viral target RNA molecules.

In a preferred embodiment, the novel system refers to a system as defined above, wherein said at least one nucleotide sequence encoding said at least one Cas13 protein and said at least one gRNA or said at least one nucleotide sequence encoding said at least one gRNA are on the same nucleotide construct of said system, the construct which further comprises said viral 5′ UTR or said nucleotide sequence encoding said 5′ UTR and/or said viral 3′ UTR or said nucleotide sequence encoding said viral 3′ UTR, wherein said viral replicase recognition sequence is comprised in at least any one of said 5′ UTR or in the nucleotide sequence encoding said 5′ UTR, or said 3′ UTR or in the nucleotide sequence encoding said 3′ UTR. Such system refers to variant 1 (see FIG. 5).

In another preferred embodiment, the novel system refers to a system as defined above in paragraph 12, wherein said at least one gRNA or said at least one nucleotide sequence encoding said at least one gRNA is split in two parts and wherein i) one part is integrated at the 5′ end of said at least one nucleotide sequence encoding said at least one Cas13 protein; and ii) the other part of said gRNA or said nucleotide sequence encoding said gRNA is integrated at the 3′ end of said at least one nucleotide sequence encoding said at least one Cas13 protein. Such system refers to variant 4 (see FIG. 8).

In another preferred embodiment, the novel system refers to a system as defined above in paragraph 11, wherein said at least one nucleotide sequence encoding said at least one Cas13 protein and said at least one gRNA or said at least one nucleotide sequence encoding said at least one gRNA are on two different nucleotide constructs of said system: the first construct comprising said at least one nucleotide sequence encoding said at least one Cas13 protein further comprises said viral 5′ UTR or said nucleotide sequence encoding said 5′ UTR and/or said viral 3′ UTR or said nucleotide sequence encoding said viral 3′ UTR, wherein said viral replicase recognition sequence is comprised in at least any one of said 5′ UTR or in the nucleotide sequence encoding said 5′ UTR, or said 3′ UTR or in the nucleotide sequence encoding said 3′ UTR; and the second construct comprising said at least one gRNA or said at least one nucleotide sequence encoding said at least one gRNA. Such system refers to variant 3 (see FIG. 7). In a preferred embodiment, such novel system as defined in paragraph 14 is comprised by the invention, wherein said gRNA of said second construct comprises the replacement of the 5′ and/or 3′ terminal nucleotides by 2′-O-methyl-3′P-thioate.

In another preferred embodiment, the novel system refers to a system as defined above in paragraph 14, wherein the second construct comprising said at least one gRNA or said at least one nucleotide sequence encoding said at least one gRNA also comprises said viral 5′ UTR or said nucleotide sequence encoding said 5′ UTR and/or said viral 3′ UTR or said nucleotide sequence encoding said viral 3′ UTR, wherein said viral replicase recognition sequence is comprised in at least any one of said 5′ UTR or in the nucleotide sequence encoding said 5′ UTR, or said 3′ UTR or in the nucleotide sequence encoding said 3′ UTR. Such system refers to variant 2 (see FIG. 6).

Additionally, the present invention may also comprise said systems as defined elsewhere herein, wherein said systems further comprise at least one nucleotide sequence encoding at least one viral packaging signal which is comprised by said nucleotide construct of said systems comprising said at least one nucleotide sequence encoding said at least one Cas13 protein (see FIGS. 5B, 6B—first construct—7B—first construct, and 8B).

Also described herein may be the systems as defined above, wherein said at least one nucleotide sequence encoding said at least one viral packaging signal is also comprised by said construct comprising said at least one gRNA or said at least one nucleotide sequence encoding said at least one gRNA as defined above in paragraph 15 (see FIG. 6B—second construct).

Encompassed herein may also be the systems as defined elsewhere herein, wherein said Cas13 protein is a Cas 13d protein.

Further encompassed herein may also be the systems as defined elsewhere herein, wherein said Cas 13d protein is derived from the genus of Ruminococcus, preferably from Ruminococcus flavevaciens.

Also described herein may be the systems as defined elsewhere herein, wherein said at least one nucleotide sequence encoding said at least one Cas13 protein
i) is fused with at least one nucleotide sequence encoding at least one nuclear localization signal (NLS) fused to at least one nucleotide sequence encoding at least one nuclear export sequence (NES), or
ii) is fused with at least one nucleotide sequence encoding at least one NLS or with at least one nucleotide sequence encoding at least one NES and wherein said nucleotide sequence encoding said gRNA is fused with at least one nucleotide sequence encoding at least one viral export element. Also described herein may be the system as defined above, wherein said at least one nucleotide sequence encoding said at least one Cas13 protein
i) is fused with at least one nucleotide sequence encoding said at least one NLS fused to at least one nucleotide sequence encoding said at least one NES, or
ii) is fused with at least one nucleotide sequence encoding said at least one NLS or with at least one nucleotide sequence encoding said at least one NES,
via a nucleotide sequence encoding a peptide linker.

Also described herein may be the system as defined above under i), wherein said at least one nucleotide sequence encoding said at least one Cas13 protein is fused with two nucleotide sequences both encoding a NLS fused to one nucleotide sequence encoding a NES. Also described herein may be the system as defined above, wherein the two NLS encoded by said two nucleotide sequences comprise the SV40 NLS having the amino acid sequence as depicted in SEQ ID NO: 5 and the NUS consensus sequence having the amino acid sequence as depicted in SEQ ID NO: 3 and the one NES encoded by said one nucleotide sequence comprises the HIV NES having the amino acid sequence as depicted in SEQ ID NO: 4.

Also described herein may be the systems as defined elsewhere herein, wherein said gRNA has a length of at least about 23 nucleotides. Also described herein may be the systems as defined elsewhere herein, wherein said gRNA has a length of between about 26 to about 30 nucleotides. Also described herein may be the systems as defined elsewhere herein, wherein said gRNA has at least about 68% complementary sequence identity to said one or more viral target RNA molecules. Also described herein may be the systems as defined elsewhere herein, wherein said gRNA is capable of hybridizing to (a) 5′- and/or 3′-untranslated region(s) of said one or more viral target RNA molecules.

The present invention may also disclose the systems as defined elsewhere herein, wherein said nucleotide sequence encoding said gRNA is fused with a tRNA or a ribozyme. In preferred embodiments, the present invention comprises that said nucleotide sequence encoding said gRNA is fused with at least one nucleotide sequence encoding at least one viral export element. Even more preferably, said at least one viral export element is a constitutive transport element (CTE) or adenovirus VA1 RNA (VARdm).

Also described herein may be the systems as defined elsewhere herein, wherein said systems inactivate viral ssRNA.

In a second aspect, the present invention also relates to a delivery system comprising the novel systems as defined elsewhere herein.

In a third aspect, the present invention also relates to a composition comprising the novel systems as defined elsewhere herein or the delivery system as defined herein. Preferably, the composition as defined herein, further comprises at least one pharmaceutically acceptable carrier.

In a fourth aspect, the present invention also relates to the novel systems or the composition for use in therapy. Further, the present invention relates to the novel systems or the composition for use in a method of preventing or treating a viral disease in a subject, the method comprising administering to the subject a therapeutically effective amount of the systems or the composition as defined herein. Preferably, the viral disease is caused by a RNA virus. Even more preferably, wherein the viral disease is any one of a coronavirus disease, influenza A, ebola, measles, hepatitis C, tick-borne encephalitis (TBE), Venezuelan Equine Encephalitis (VEE) viral infection, dengue fever, yellow fever, bunya virus disease, respiratory syncytial virus (RSV) disease or zika fever, most preferably wherein the viral disease is the COVID-19 disease.

In a fifth aspect, the present invention also relates to a kit comprising the novel systems as defined herein. Preferably, the kit further comprises a delivery system as defined herein and/or a label.

Finally, in a sixth aspect, the present invention also relates to a method of producing the novel systems as defined elsewhere herein, the method comprising a) synthesizing said at least one nucleotide sequence encoding said at least one Cas13 protein, said at least one gRNA or said at least one nucleotide sequence encoding said at least one gRNA and said viral 5′ UTR or said nucleotide sequence encoding said 5′ UTR and/or said viral 3′ UTR or said nucleotide sequence encoding said 3′ UTR by means of genetic engineering methods, thereby producing said system; optionally b) obtaining said produced system of step a).

### BRIEF DESCRIPTION OF THE FIGURES

FIG. 1: Illustration of co-replication and packaging system. The construct is derived from viral elements: Viral 5′ and 3′ UTRs are recognized by the viral polymerase and the viral packaging signal enables packaging of Cas13 in viral particles. Upon entry, the viral polymerase replicates the viral genome and in parallel the CRISPR/Cas13 system. The co-replicated Cas13 system gets packaged in virus-derived particles and spreads to viral target cell types. Demonstrated is the use of a sense strand nucleotide sequence.

FIG. 2: Theoretical model of co-replicating Cas13 impacting the viral load. Non-replicating Cas13 (2) reduces the viral load but cannot inhibit the viral replication below the lethal viral load. Addition of co-replication elements to Cas13 inhibits viral replication below the lethal threshold (3).

FIG. 3: Cas13 constructs (referring to variant 3, see FIG. 7), flanked by viral replication elements and extended by an mRuby3 fluorescent reporter protein, enables co-replication of Cas13 along with the reporter protein. Increase of mRuby3 intensity in co-replication construct was measured over 72 h in SARS-Cov-2 infected cells.

FIG. 4: Inhibition of SARS-CoV-2 replication in cells expressing the Cas13 co-replication system along with a gRNA against the virus (round), compared to a co-replication system with a non-target gRNA (square). SARS-CoV-2 replication was live measures for 72 h by fluorescence intensity of a virus encoded GFP.

FIG. 5: Cas13 co-replication system—variant 1—is a single RNA or DNA molecule, composed of the Cas13 coding sequence and a gRNA, which is complementary to the virus, or nucleotide sequences encoding the Cas13 protein and the gRNA. The system comprises one construct with viral 5′ and 3′ UTRs, wherein the construct is flanked by a viral replication element, the viral replicase recognition sequence, within said 5′ and said 3′ UTRs, and therefore co-replicates with the virus in trans. Cas13 co-replication systems either without (A) or with (B) the viral packaging signal. Demonstrated is the use of a sense strand nucleotide sequence.

FIG. 6: Cas13 co-replication system—variant 2—comprises two constructs, wherein it separates the Cas13 coding sequence from the gRNA on two independent RNA molecules. The same applies mutatis mutandis to such system on DNA level. Both constructs comprising viral 5′ and 3′ UTRs are flanked by the viral replication element, the viral replicase recognition sequence, within said 5′ and said 3′ UTRs, and therefore co-replicate with the virus. Cas13 co-replication systems either without (A) or with (B) the viral packaging signal. Demonstrated is the use of a sense strand nucleotide sequence.

FIG. 7: In Cas13 co-replication system—variant 3—only the Cas13 coding mRNA co-replicates while the gRNA is supplemented, but co-replication incompetent. This system variant also comprises two constructs (see FIG. 6), wherein only the construct of the Cas13 coding mRNA comprises viral 5′ and 3′ UTRs and is thus flanked by the viral replication element, the viral replicase recognition sequence, within said 5′ and said 3′ UTRs. The same applies mutatis mutandis to such system on DNA level. Cas13 co-replication systems either without (A) or with (B) the viral packaging signal. Demonstrated is the use of a sense strand nucleotide sequence.

FIG. 8: Cas13 co-replication system—variant 4—splits the gRNA into two parts by generating an artificial TRS-B/-L sequence, inducing discontinuous transcription and resulting in a sub-genomic mRNA coding for a bona fide gRNA. The system comprises again only one construct comprising viral 5′ and 3′ UTRs (see again FIG. 5), wherein the construct is flanked by the viral replication element, the viral replicase recognition sequence, within said 5′ and said 3′ UTRs, and therefore co-replicates with the virus. The same applies mutatis mutandis to such system on DNA level. Cas13 co-replication systems either without (A) or with (B) the viral packaging signal. Demonstrated is the use of a sense strand nucleotide sequence.

FIG. 9: Knockdown efficiency of different Cas13 protein und gRNA variants. Each variant was targeted against a co-transfected firefly luciferase and normalized to a non-target gRNA. The fusion of a NLS-NES tandem signal to Cas13d improved the efficiency compared to the NLS-only variant. Elongation of the gRNA further improved the efficiency. All conditions were compared to the latest generation of miRNAs/RNA interference (Fellmann et al., 2013 in Cell reports).

FIG. 10: Characterization of different gRNA lengths for Cas13d-NLS-NES for targeting a co-transfected nanoluciferase. The optimal length of the gRNA is in the range of 26 bp 30 bp.

FIG. 11: Different strategies to export the gRNA from the nucleus to the cytosol are compared. Knockdown efficiencies against a co-transfected nanoluciferase were measured for different Cas13d protein localizations and different gRNA expression strategies. Polymerase III driven gRNAs remain in the nucleus. Therefore, nuclear localized Cas13d (NLS) is superior to cytosolic Cas13d (NES). A tandem fusion (NLS-NES) maximizes the knockdown efficiency, because of picking up the gRNA in the nucleus and being active against the cytosolic mRNA coding for Nanoluciferase. Western Blot confirms that a gRNA being present in the same compartment as Cas13d, stabilizes the protein. A complementary strategy to export the gRNA by expression from a Polymerase II driven promoter does not improve the efficiency.

FIG. 12: Knockdown efficiency of differentially localized Cas13d variants against co-transfected nanoluciferase target RNA (crRNA 1 and crRNA 2). The balanced set of nuclear and cytosolic localization signals of Cas13d maximises the knockdown efficiency for two crRNAs.

FIG. 13: (A) Representative images showing the localization of Cas13d protein variants, fused to different localization signals. (B) Quantification of cytosolic/nuclear distribution of different Cas13d variants for 100 cells.

FIG. 14: Comparison of knockdown efficiencies for two gRNAs targeting a co-transfected nanoluciferase for NES or tandem NLS-NES fused to Cas13. Additionally, either an unmodified, or CTE fused gRNA was tested. The combination of NLS-NES and CTE-gRNA maximizes the knockdown efficiency.

FIG. 15: (A) Illustration of different crRNA export strategies. Polymerase III (pol III) driven crRNAs remain in the nucleus, polymerase II (pol II) driven crRNA are exported to the cytosol, crRNAs fused to are constitutive transport element (CTE) are exported, as well as crRNAs fused to a minihelix of adenovirus VA1 RNA (VARdm). (B) Comparison of knockdown efficiencies against nanoluciferase target RNA (crRNA 1 and crRNA 2) for different crRNA export motifs together with either cytosolic (NES) or nuclear (NLS) Cas13d protein. CrRNAs fused to a CTE export motif is most efficient in driving Cas13d based knockdown in the cytosol.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments described throughout the specification should not be construed to limit the present invention to only the explicitly described embodiments. This description should be understood to support and encompass embodiments which combine the explicitly described embodiments with any number of the disclosed and/or preferred elements. Furthermore, any permutations and combinations of all elements described herein should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. When used herein the term “comprising” can be substituted with the term “containing” or “including” or sometimes when used herein with the term “having”. When used herein “consisting of” excludes any element, step, or ingredient not specified.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless otherwise indicated, the term “at least” preceding a series of elements is to be understood to refer to every element in the series. The term “at least one” refers to one, two, three or more such as four, five, six, seven, eight, nine, ten and more. Those skilled in the art will recognize, or be able to ascertain using no more than routine experimentation, many equivalents to the specific embodiments of the invention described herein. Such equivalents are intended to be encompassed by the present invention.

The term “less than” or in turn “more than” or “below” does not include the concrete number.

The term “and/or” wherever used herein includes the meaning of “and”, “or” and “all or any other combination of the elements connected by said term”.

When used herein “consisting of” excludes any element, step, or ingredient not specified in the claim element. When used herein, “consisting essentially of” does not exclude materials or steps that do not materially affect the basic and novel characteristics of the claim.

The term “including” means “including but not limited to”. “Including” and “including but not limited to” are used interchangeably.

The term “about” means plus or minus 20%, preferably plus or minus 10%, more preferably plus or minus 5%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the examples, offered for illustrative purposes only. The examples are not intended to limit the scope of the present invention in any way.

### Systems

The present invention refers to a CRISPR system which stands for “clustered, regularly interspaced, short palindromic repeats (CRISPR)/CRISPR-associated protein”. It is based on an adaptive defense mechanism evolved by bacteria and archaea to protect them from invading viruses, bacteria and plasmids, which relies on small RNAs for sequence-specific detection and silencing of foreign ribonucleic acids. CRISPR/Cas systems are composed of Cas genes organized in operon(s) and CRISPR array(s) consisting of genome-targeting sequences (called spacers) interspersed with identical repeats (Bhaya et al. (2011) Annu Rev Genet 45:273-297; Barrangou R and Horvath P (2012) Annu Rev Food Sci Technol 3:143-162). Target recognition by guide RNAs (gRNAs) directs the silencing of the foreign sequences by means of Cas proteins that function in complex with the gRNAs.

In more detail, a CRISPR-Cas or CRISPR system refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the present invention the novel system refers to a CRISPR-Cas13 system.

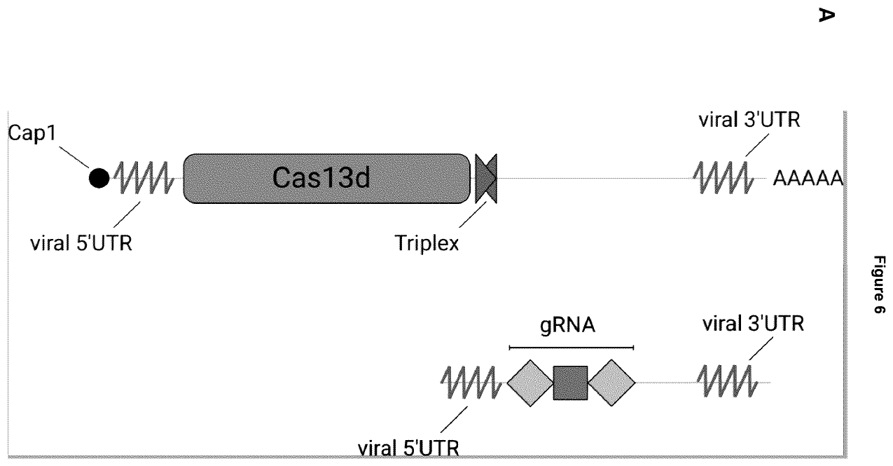

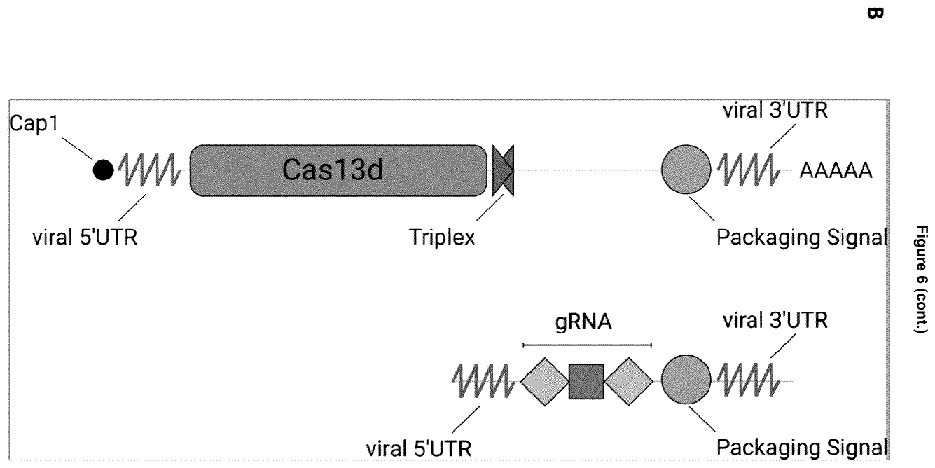

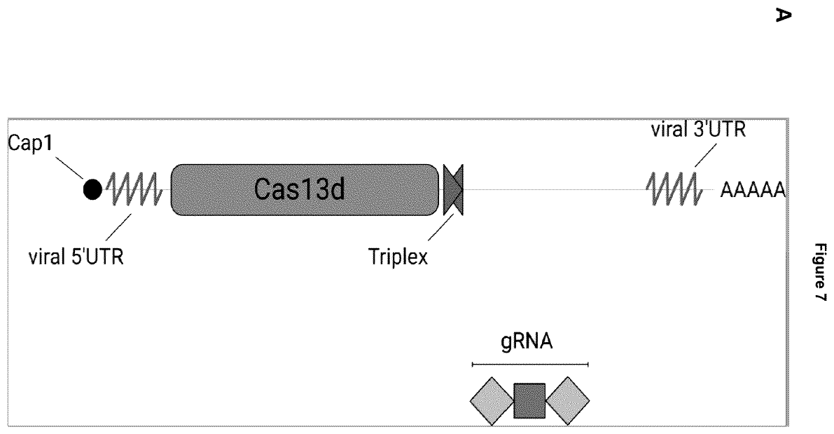

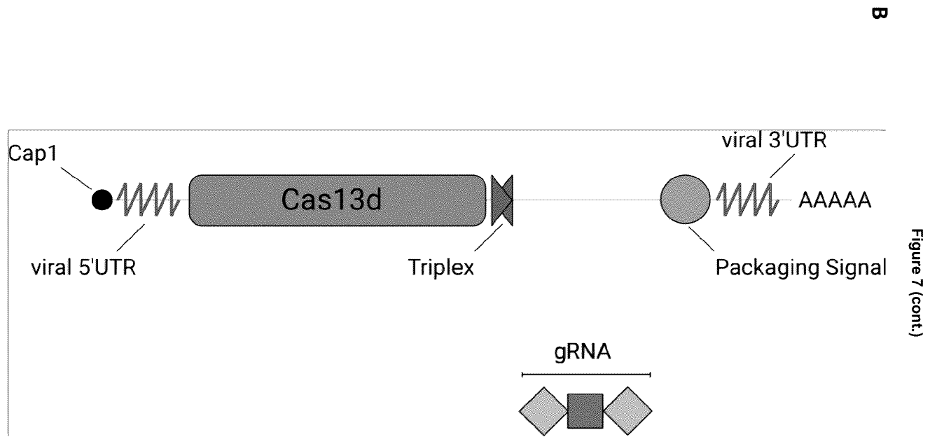

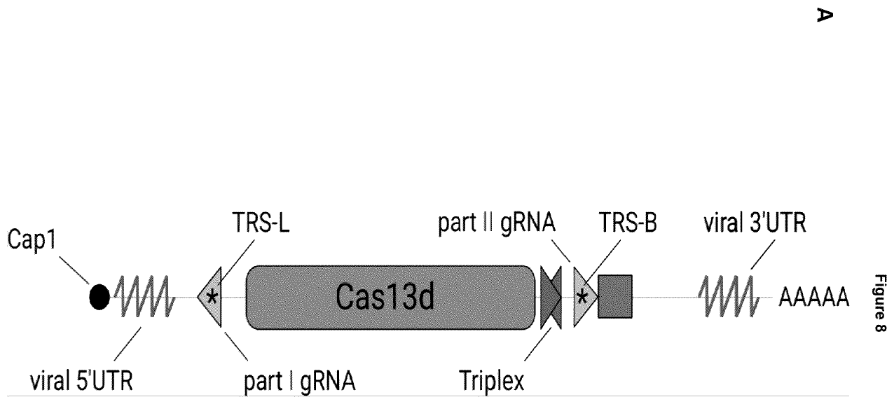

In the present invention said systems as will be defined herein comprise at least one (one or more) nucleotide sequence encoding at least one clustered, regularly interspaced, short palindromic repeats (CRISPR)-associated (Cas) protein (short: Cas13 protein). Thus, the systems comprise at least one (one or more) such as one, two, three, four, five or more nucleotide sequences encoding at least one Cas13 protein as defined elsewhere herein. Such systems may comprise one nucleotide construct comprising at least one nucleotide sequence encoding at least one Cas13 protein and comprising at least one gRNA or at least one nucleotide sequence encoding said at least one gRNA as will be defined elsewhere herein when characterizing the four different system variants (see FIGS. 5 and 8). When said systems comprise two nucleotide constructs, then the at least one nucleotide sequence encoding at least one Cas13 protein is comprised by one nucleotide construct and the at least one gRNA or the at least one nucleotide sequence encoding said at least one gRNA is comprised by another nucleotide construct as will be defined elsewhere herein (see FIGS. 6 and 7). CRISPR-Cas13 is an RNA targeting and editing system based on the bacterial immune system that protects them from viruses and bacteria. Cas13 is the effector protein that targets and cleaves invading ribonucleic acids from viruses and bacteria in type VI CRISPR-Cas systems. The CRISPR-Cas13 system is analogous to the CRISPR-Cas9 system. However, unlike Cas9 which targets DNA, Cas13, an RNAse, targets/detects and cleaves/degrades single stranded RNA (ssRNA). Thus, a Cas13 protein also refers to a (Type VI) RNA-targeting effector protein. Cas13 enzymes have two higher eukaryotes and prokaryotes nucleotide-binding (HEPN) endoRNase domains that mediate precise RNA cleavage with a preference for targets with protospacer flanking sites (PFSs) observed biochemically and in bacteria. Example RNA-targeting effector proteins include C2c2 (now known as Cas13a), Cas13b, Cas13c and Cas13d. Cas13 was first discovered in L. shahii, a species of the Leptotrichia bacteria while researchers were looking for previously unidentified CRISPR systems. Since Cas13 proteins were identified in 2016 by the research group of Feng Zhang (Broad Institute, MIT), four different subtypes (Cas13a-d) have been described and intensively studied so far. They differ strongly in their sequences, but have as a common feature so-called two HEPN domains (Higher Eukaryotes and Prokaryotes Nucleotide Binding Domains), which are responsible for RNAse activity. Such domains of said Cas13 protein mediate precise RNA cleavage with a preference for targets with protospacer flanking sites (PFSs).

Since said systems of the present invention target RNA molecules, wherein the at least one Cas13 protein encoded by the at least one nucleotide sequence forms a complex with the at least one gRNA encoded by the at least one nucleotide sequence and wherein the at least one gRNA directs the complex to the one or more target RNA molecules, thereby targeting the one or more target RNA molecules, said CRISPR system may also refer to a ribonucleic acid detection system. Since said systems not only target RNA molecules via said Cas13 RNAse, but then also cleave said target RNA molecules, thereby degrading said RNA molecules, said CRISPR system may also refer to a ribonucleic acid degradation system.

The systems of the present invention comprising either a viral 5′ UTR or a nucleotide sequence encoding said 5′ UTR or a viral 3′ UTR or a nucleotide sequence encoding said viral 3′ UTR or both, comprise said UTR(s) from a virus as defined herein which is the same virus that the replicase recognition sequence is from. Said viral 5′ and/or 3′ UTRs are used i) due to their stabilizing function, ii) providing longevity of said nucleotide constructs of said system, iii) initiating translation due to the binding of the ribosome to the 5′ UTR and iv) due to their viral transcription enabling functions, and/or v) providing at least a viral recognition sequence for replication, transcription and translation initiation. Thus, said viral 5′ and/or 3′ UTRs comprise functional elements such as regulatory elements for the replication, transcription and translation of the viral genome and the viral sub-genomic RNA (sgRNA) as known to a person skilled in the art. Such elements refer but are not limited to promoter region(s) including 5′ non-coding sequences involved in initiation of transcription and translation, which may also include enhancer or repressor elements as well as translated signal and leader sequences for targeting the native polypeptide to a specific compartment of a host cell, if necessary; a translation initiation codon, transcriptional and translational enhancers, and/or an origin of replication.

Said viral replicase recognition sequence can be comprised/located either in said viral 5′ UTR or in the nucleotide sequence encoding said viral 5′ UTR, e.g. if the system only comprises said viral 5′ UTR or said nucleotide sequence encoding said 5′ UTR, or in said viral 3′ UTR or in the nucleotide sequence encoding said viral 3′ UTR, e.g. if the system only comprises said viral 3′ UTR or said nucleotide sequence encoding said 3′ UTR; or in both, e.g. if the system comprises both or even more viral UTRs. Even if the system comprises both viral UTRs, said viral replicase recognition sequence can be comprised either in said viral 5′ UTR or in the nucleotide sequence encoding said viral 5′ UTR or in said viral 3′ UTR or in the nucleotide sequence encoding said viral 3′ UTR. During replication, the genomic RNA (also called gRNA) is cyclated via the base pairing of 5′ and 3′ UTRs. This then serves as a matrix for the synthesis of the (−) strand (−) RNA), which in turn serves as the basis for the generation of the (+) strand ((+) RNA). A “viral replicase recognition sequence” refers to a conserved sequence/replication element of the viral genome and is a signal sequence for the viral replicase which recognizes said sequence. Such sequence is located in the viral UTR(s) (either in the 5′ UTR, the 3′ UTR or in both) as mentioned elsewhere herein. As already mentioned herein, each system variant does not comprise a nucleotide sequence encoding a viral replicase. Thus, a viral replicase not being encoded by the system of the present invention, but present in the cells of the subject after said virus has infected/will infect said subject to which the system is delivered to with its genome and after translation in the subject, is able to recognize the encoded viral replicase recognition sequence(s) comprised in said UTR(s) of said systems of the present invention. Thus, the viral replicase recognition sequence(s) being recognized by said viral replicase is/are also propagated in the course of virus replication by the virus's own machinery. A “viral replicase” refers to any viral enzyme (also called RNA-dependent RNA polymerase (RdRp) or RNA replicase) which catalyzes the replication of RNA from an RNA template as known to a person skilled in the art. Such replicase can be from any virus as defined herein which has infected/will infect a subject as defined herein and to which said system comprising said viral replicase recognition sequence(s) comprised in said UTR(s) selected according to the infecting virus, of the present invention is then delivered to. Preferably, a subject to which the system of the invention is delivered to has been infected with a coronavirus, influenza A virus, ebola virus, morbilivirus, hepacivirus, flavivirus such as TBE virus, venezuelan equine encephalitis virus, dengue virus, yellow fever virus or zika virus so that the viral replicase capable of recognizing said viral replicase recognition sequence is from the virus as mentioned (e.g. coronavirus) which has infected said subject. Having said that, also the viral replicase recognition sequence being comprised within said UTR(s) of said systems is from the same virus the subject has been infected with (e.g. coronavirus) and from which the viral replicase recognizing the recognition sequence is from (e.g. coronavirus). In other words, the viral replicase recognition sequence being comprised within the system of the invention is from the same virus (e.g. coronavirus) as the one or more viral target RNA molecules of the RNA virus (e.g. coronavirus) that has infected the subject. The load of the viral agent is thus automatically adapted directly to the viral load by means of internal dose adjustment. In sum, the novel systems of the present invention which comprise said replication element(s) multiply depending on the viral load and lead to complete inhibition of virus replication. Said systems can thus be applied as virus agent against preferably any RNA virus as defined herein.

The system variant 1 which is depicted in FIG. 5A refers to one nucleotide construct comprising at least one nucleotide sequence encoding at least one Cas13 protein and comprising at least one gRNA or at least one nucleotide sequence encoding said at least one gRNA as well as comprising a viral 5′ UTR or a nucleotide sequence encoding said 5′ UTR and/or a viral 3′ UTR or a nucleotide sequence encoding said viral 3′ UTR, wherein said viral replicase recognition sequence is comprised either in said 5′ UTR or in the nucleotide sequence encoding said 5′ UTR, or in said 3′ UTR or in the nucleotide sequence encoding said 3′ UTR or in both, preferably comprising both UTRs or nucleotide sequences encoding said UTRs, wherein said viral replicase recognition sequence is comprised in said 5′ UTR or in the nucleotide sequence encoding said 5′ UTR and in said 3′ UTR or in the nucleotide sequence encoding said 3′ UTR. Here, a co-replication of the whole nucleotide construct system together with the replication of the infecting virus is achieved, providing a self dosing mechanism according to the viral load. In one embodiment said novel system variant 1 comprises one nucleotide construct as defined above, wherein the nucleotide construct refers to a sense (+) single strand nucleotide sequence on the DNA or RNA level, wherein the nucleotide construct comprises at least said embodiment of a viral 5′ UTR or said nucleotide sequence encoding said 5′ UTR and wherein said viral replicase recognition sequence is located/comprised within said viral 5′ UTR or said nucleotide sequence encoding said 5′ UTR. In a preferred embodiment said novel system variant 1 comprises one nucleotide construct as defined above, wherein the nucleotide construct refers to an antisense (−) single strand nucleotide sequence on the DNA or RNA level, wherein the nucleotide construct comprises at least said embodiment of a viral 3′ UTR or said nucleotide sequence encoding said 3′ UTR and wherein said viral replicase recognition sequence is located/comprised within said 3′ UTR or said nucleotide sequence encoding said 3′ UTR. In another embodiment said novel system variant 1 comprises one nucleotide construct as defined above, wherein the nucleotide construct refers to a sense (+) single strand nucleotide sequence on the DNA or RNA level, wherein the nucleotide construct comprises both said viral 3′ UTR or said nucleotide sequence encoding said 3′ UTR, and said viral 5′ UTR or said nucleotide sequence encoding said 5′ UTR and wherein said viral replicase recognition sequence is located/comprised within at least one of said viral UTRs or said nucleotide sequence(s) encoding said viral UTR(s) (in said 5′ UTR or in said 3′ UTR or in both). In an even more preferred embodiment said novel system variant 1 comprises one nucleotide construct as defined above, wherein the nucleotide construct refers to an antisense (−) single strand nucleotide sequence on the DNA or RNA level, wherein the nucleotide construct comprises both said viral 3′ UTR or said nucleotide sequence encoding said 3′ UTR, and said viral 5′ UTR or said nucleotide sequence encoding said 5′ UTR and wherein said viral replicase recognition sequence is located/comprised within at least one of said viral UTRs or said nucleotide sequence(s) encoding said viral UTR(s) (in said 5′ UTR or in said 3′ UTR or in both). The application of antisense (−) single strand nucleotide sequence as nucleotide construct of system variant 1 leads to a Cas13 protein production only when a viral replicase is already present in the cell after a previous infection of the subject. This is due to the fact that the antisense (−) strand cannot be directly used by the ribosome for protein synthesis as it would be the case with a sense (+) single strand nucleotide sequence as nucleotide construct of system variant 1. In cells not being infected with the virus, no Cas13 protein is produced, since the replicase firstly needs to generate the sense (+) strand. Further, any anticipated side effects can be completely excluded by marginal amounts of Cas13 (as it would be in a direct production of the sense (+) single strand).

The system variant 2 which is depicted in FIG. 6A refers to two nucleotide constructs, wherein the first construct comprises said at least one nucleotide sequence encoding said at least one Cas13 protein as well as said viral 5′ UTR or said nucleotide sequence encoding said 5′ UTR and/or said viral 3′ UTR or said nucleotide sequence encoding said viral 3′ UTR, wherein said viral replicase recognition sequence is comprised either in said 5′ UTR or in the nucleotide sequence encoding said 5′ UTR, or in said 3′ UTR or in the nucleotide sequence encoding said 3′ UTR or in both, preferably comprising both UTRs or nucleotide sequences encoding said UTRs, wherein said viral replicase recognition sequence is comprised in said 5′ UTR or in the nucleotide sequence encoding said 5′ UTR and in said 3′ UTR or in the nucleotide sequence encoding said 3′ UTR; and the second construct comprising said at least one gRNA or said nucleotide sequence encoding said at least one gRNA also comprises said viral 5′ UTR or said nucleotide sequence encoding said 5′ UTR and/or said viral 3′ UTR or said nucleotide sequence encoding said viral 3′ UTR, again wherein said viral replicase recognition sequence is comprised either in said 5′ UTR or in the nucleotide sequence encoding said 5′ UTR, or in said 3′ UTR or in the nucleotide sequence encoding said 3′ UTR or in both, preferably comprising both UTRs or nucleotide sequences encoding said UTRs, wherein said viral replicase recognition sequence is comprised in said 5′ UTR or in the nucleotide sequence encoding said 5′ UTR and in said 3′ UTR or in the nucleotide sequence encoding said 3′ UTR. Preferably, the location of said viral replicase recognition sequence within the second construct of system variant 2 depends on the location of said viral replicase recognition sequence within the first construct. If for example the recognition sequence is comprised in said viral 5′ UTR or in said nucleotide sequence encoding said 5′ UTR of said first construct, also the recognition sequence of the second construct is comprised within said viral 5′ UTR or in said nucleotide sequence encoding said 5′ UTR. Here, a co-replication of the two separate nucleotide construct systems together with the replication of the infecting virus is achieved. For this system variant 2 the dosage of each construct during application may differ and a separate production of the components of each construct can be achieved. In addition, the gRNA is processed out of the system variant. Once this happens, the UTRs are cut off and replication of the gRNA no longer takes place. If both elements (Cas13 and gRNA) are separated from each other as it is the case for system variant 2, at least the Cas13 protein is further replicated and amplified. Thus, not only the first construct, but also the second construct is replicated by the virus due to both constructs comprising said viral replicase recognition sequence. In one embodiment said novel system variant 2 comprises two nucleotide constructs as defined above, wherein both nucleotide constructs refer to a sense (+) single strand nucleotide sequence on the DNA or RNA level, wherein each of the nucleotide constructs comprise at least said embodiment of a viral 5′ UTR or said nucleotide sequence encoding said 5′ UTR and wherein said viral replicase recognition sequence is located/comprised within said viral 5′ UTR or said nucleotide sequence encoding said 5′ UTR of each nucleotide constructs. In a preferred embodiment and based on the advantage of the application of an antisense strand as defined above, said novel system variant 2 comprises two nucleotide constructs as defined herein, wherein both nucleotide constructs refer to an antisense (−) single strand nucleotide sequence on the DNA or RNA level, wherein each of the nucleotide constructs comprise at least said embodiment of a viral 3′ UTR or said nucleotide sequence encoding said 3′ UTR and wherein said viral replicase recognition sequence is located/comprised within said viral 3′ UTR or said nucleotide sequence encoding said 3′ UTR. In another embodiment said novel system variant 2 comprises two nucleotide constructs as defined above, wherein both nucleotide constructs refer to a sense (+) single strand nucleotide sequence on the DNA or RNA level, wherein each of the nucleotide constructs comprise both said viral 3′ UTR or said nucleotide sequence encoding said 3′ UTR, and said viral 5′ UTR or said nucleotide sequence encoding said 5′ UTR and wherein said viral replicase recognition sequence is located/comprised within at least one of said viral UTRs or said nucleotide sequence(s) encoding said viral UTR(s) (in said 5′ UTR or in said 3′ UTR or in both). In an even more preferred embodiment and based on the advantage of the application of an antisense strand as defined above, said novel system variant 2 comprises two nucleotide constructs as defined above, wherein both nucleotide constructs refer to an antisense (−) single strand nucleotide sequence on the DNA or RNA level, wherein each of the nucleotide constructs comprise both said viral 3′ UTR or said nucleotide sequence encoding said 3′ UTR, and said viral 5′ UTR or said nucleotide sequence encoding said 5′ UTR and wherein said viral replicase recognition sequence is located/comprised within at least one of said viral UTRs or said nucleotide sequence(s) encoding said viral UTR(s) (in said 5′ UTR or in said 3′ UTR or in both).

The system variant 3 which is depicted in FIG. 7A refers to two nucleotide constructs, wherein the first construct comprises said at least one nucleotide sequence encoding said at least one Cas13 protein as well as said viral 5′ UTR or said nucleotide sequence encoding said 5′ UTR and/or said viral 3′ UTR or said nucleotide sequence encoding said viral 3′ UTR, wherein said viral replicase recognition sequence is comprised either in said 5′ UTR or in the nucleotide sequence encoding said 5′ UTR, or in said 3′ UTR or in the nucleotide sequence encoding said 3′ UTR or in both, preferably comprising both UTRs or nucleotide sequences encoding said UTRs, wherein said viral replicase recognition sequence is comprised in said 5′ UTR or in the nucleotide sequence encoding said 5′ UTR and in said 3′ UTR or in the nucleotide sequence encoding said 3′ UTR; and the second construct comprising said at least one gRNA or said nucleotide sequence encoding said at least one gRNA without any UTR sequences comprised herein. Here, a co-replication of only the first nucleotide construct comprising said nucleotide sequence encoding said Cas13 protein together with the replication of the infecting virus is achieved, but no co-replication of the second construct comprising the gRNA or the nucleotide sequence encoding said gRNA. This has the advantage that the second construct comprising the gRNA or the nucleotide sequence encoding said gRNA does not suffer from any loss of stability due to repeated replication cycles. If both elements (Cas13 or gRNA) are separated from each other, at least the Cas13 protein is further replicated and amplified. If needed, additional gRNA can be administered. In one embodiment said novel system variant 3 comprises two nucleotide constructs as defined above, wherein both nucleotide constructs refer to a sense (+) single strand nucleotide sequence on the DNA or RNA level, wherein only the first nucleotide construct comprises at least said embodiment of a viral 5′ UTR or said nucleotide sequence encoding said 5′ UTR and wherein said viral replicase recognition sequence is located/comprised within said viral 5′ UTR or said nucleotide sequence encoding said 5′ UTR of the first nucleotide construct. In a preferred embodiment and based on the advantage of the application of an antisense strand as defined above, said novel system variant 3 comprises two nucleotide constructs as defined herein, wherein both nucleotide constructs refer to an antisense (−) single strand nucleotide sequence on the DNA or RNA level, wherein only the first nucleotide construct comprises at least said embodiment of a viral 3′ UTR or said nucleotide sequence encoding said 3′ UTR and wherein said viral replicase recognition sequence is located/comprised within said viral 3′ UTR or said nucleotide sequence encoding said 3′ UTR. In another embodiment said novel system variant 3 comprises two nucleotide constructs as defined above, wherein both nucleotide constructs refer to a sense (+) single strand nucleotide sequence on the DNA or RNA level, wherein only the first nucleotide construct comprises both said viral 3′ UTR or said nucleotide sequence encoding said 3′ UTR, and said viral 5′ UTR or said nucleotide sequence encoding said 5′ UTR and wherein said viral replicase recognition sequence is located/comprised within at least one of said viral UTRs or said nucleotide sequence(s) encoding said viral UTR(s) (in said 5′ UTR or in said 3′ UTR or in both). In an even more preferred embodiment and based on the advantage of the application of an antisense strand as defined above, said novel system variant 3 comprises two nucleotide constructs as defined above, wherein both nucleotide constructs refer to an antisense (−) single strand nucleotide sequence on the DNA or RNA level, wherein only the first nucleotide construct comprises both said viral 3′ UTR or said nucleotide sequence encoding said 3′ UTR, and said viral 5′ UTR or said nucleotide sequence encoding said 5′ UTR and wherein said viral replicase recognition sequence is located/comprised within at least one of said viral UTRs or said nucleotide sequence(s) encoding said viral UTR(s) (in said 5′ UTR or in said 3′ UTR or in both).

The system variant 4 which is depicted in FIG. 8A refers to one nucleotide construct comprising at least one nucleotide sequence encoding at least one Cas13 protein as it is the case for variant 1, wherein however said at least one gRNA or said nucleotide sequence encoding said at least one gRNA is split in two parts and wherein i) one part is integrated at the 5′ end of said at least one nucleotide sequence encoding said at least one Cas13 protein and ii) the other part of said gRNA or said nucleotide sequence encoding said gRNA is integrated at the 3′ end of said at least one nucleotide sequence encoding said at least one Cas13 protein. Additionally, said one nucleotide construct comprises a viral 5′ UTR or a nucleotide sequence encoding said 5′ UTR and/or a viral 3′ UTR or a nucleotide sequence encoding said viral 3′ UTR, wherein said viral replicase recognition sequence is comprised either in said 5′ UTR or in the nucleotide sequence encoding said 5′ UTR, or in said 3′ UTR or in the nucleotide sequence encoding said 3′ UTR or in both, preferably comprising both UTRs or nucleotide sequences encoding said UTRs, wherein said viral replicase recognition sequence is comprised in said 5′ UTR or in the nucleotide sequence encoding said 5′ UTR and in said 3′ UTR or in the nucleotide sequence encoding said 3′ UTR. Here, a co-replication of the whole nucleotide construct system together with the replication of the infecting virus is achieved, providing a self dosing mechanism according to the viral load. For system variant 4, the viral replicase generates a so-called sub-genomic mRNA by reading the two gRNA parts in combination. Only this combined gRNA sequence is then spliced and functional, i.e. it can be processed by said Cas13 protein and thus be used as a template for cutting viral target RNA molecules. The original RNA or DNA system variant 4 construct additionally comprising said gRNA or said nucleotide sequence encoding said gRNA in two parts, wherein said gRNA is—through the separation of said gRNA into two parts—not entirely complementary to the original gRNA, remains unchanged and may thus further be replicated together with the Cas13 protein. In one embodiment said novel system variant 4 comprises one nucleotide construct as defined above, wherein the nucleotide construct refers to a sense (+) single strand nucleotide sequence on the DNA or RNA level, wherein the nucleotide construct comprises at least said embodiment of a viral 5′ UTR or said nucleotide sequence encoding said 5′ UTR and wherein said viral replicase recognition sequence is located/comprised within said viral 5′ UTR or said nucleotide sequence encoding said 5′ UTR. In a preferred embodiment and based on the advantage of the application of an antisense strand as defined above, said novel system variant 4 comprises one nucleotide construct as defined above, wherein the nucleotide construct refers to an antisense (−) single strand nucleotide sequence on the DNA or RNA level, wherein the nucleotide construct comprises at least said embodiment of a viral 3′ UTR or said nucleotide sequence encoding said 3′ UTR and wherein said viral replicase recognition sequence is located/comprised within said 3′ UTR or said nucleotide sequence encoding said 3′ UTR. In another embodiment said novel system variant 4 comprises one nucleotide construct as defined above, wherein the nucleotide construct refers to a sense (+) single strand nucleotide sequence on the DNA or RNA level, wherein the nucleotide construct comprises both said viral 3′ UTR or said nucleotide sequence encoding said 3′ UTR, and said viral 5′ UTR or said nucleotide sequence encoding said 5′ UTR and wherein said viral replicase recognition sequence is located/comprised within at least one of said viral UTRs or said nucleotide sequence(s) encoding said viral UTR(s) (in said 5′ UTR or in said 3′ UTR or in both). In an even more preferred embodiment and based on the advantage of the application of an antisense strand as defined above, said novel system variant 4 comprises one nucleotide construct as defined above, wherein the nucleotide construct refers to an antisense (−) single strand nucleotide sequence on the DNA or RNA level, wherein the nucleotide construct comprises both said viral 3′ UTR or said nucleotide sequence encoding said 3′ UTR, and said viral 5′ UTR or said nucleotide sequence encoding said 5′ UTR and wherein said viral replicase recognition sequence is located/comprised within at least one of said viral UTRs or said nucleotide sequence(s) encoding said viral UTR(s) (in said 5′ UTR or in said 3′ UTR or in both). The localization of the two gRNA parts (gRNA segments) may further be independent of the sense direction of the nucleotide strand, since the gRNA parts are recognized by Cas13 and processed out from the RNA, preferably one part of the gRNA is comprised at the 5′ end of the at least one nucleotide sequence encoding at least one Cas13 protein and the other part of the gRNA at the 3′ end of said at least one nucleotide sequence encoding at least one Cas13 protein. The splitting of said gRNA into two parts when applying said system variant 4 may depend on the sequence identity of said viral gRNA or said nucleotide sequence encoding said viral gRNA as used herein such as a sequence identity of at least about 90% with the viral genome nucleotide sequences the subject is infected with, e.g. one part of the gRNA used as depicted by SEQ ID NO: 10 or the nucleotide sequence encoding SEQ ID NO: 10 has a sequence identity of at least about 90%, preferably of at least about 98% or even 100% with the TRS-L/-8 sequence of SARs-CoV-2, where the polymerase gets off at such TRS-L/-B sequence, why a splitting of said gRNA at this particular part being similar as defined herein or even identical to such TRS-U-B sequence into two parts can be performed). Depending on the viral gRNA used within said system (and thus depending on the virus the subject is infected with), the splitting of such gRNA into two parts may be at different positions within said sequence.

In one embodiment, the Cas13 protein comprises at least one HEPN domain, including but not limited to the HEPN domains known in the art, and domains recognized to be HEPN domains by comparison to consensus sequence motifs. In certain embodiments, the Cas13 protein comprises a single HEPN domain. In certain other embodiments, the Cas13 protein comprises two HEPN domains. In another embodiment, the Cas13 protein comprises one or more HEPN domains comprising a RxxxxH motif sequence. The RxxxxH motif sequence can be, without limitation, from a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In one embodiment, the at least one nucleotide sequence encoding said Cas13 protein of said systems, encodes a Cas13a protein or a functional variant thereof or a homologue or an orthologue thereof. In another embodiment, the at least one nucleotide sequence encoding said Cas13 protein of said systems, encodes a Cas13b protein or a functional variant thereof or a homologue or an orthologue thereof. In even another embodiment, the at least one nucleotide sequence encoding said Cas13 protein of said systems, encodes a Cas13c protein or a functional variant thereof or a homologue or an orthologue thereof. The Cas13a protein may be from an organism selected from the group consisting of Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter and Lachnospira or the Cas13a protein is selected from the group consisting of: Leptotrichia shahii, Leptotrichia wadei, Listeria seeligeri, Lachnospiraceae bacterium, Clostridium aminophilum, Carnobacterium gallinarum, Paludibacter propionicigenes, Listeria weihenstephanensis, Rhodobacter capsulatus, preferably from Leptotrichia wadei. In another embodiment said Cas13a protein may be selected from an amino acid sequence having at least 80% sequence identity to any of the sequences listed in Table 4 and 5 of US20200165594, which is herein incorporated by reference. In certain embodiments, Cas13b is from an organism selected from Bergeyella, Prevotella, Porphyromonas, Bacteroides, Alistipes, Riemerella, Capnocytophaga, Flavobacterium, Myroides, Chryseobacterium, Paludibacter, Psychroflexus, Phaeodactylibacter Sinomicrobium, Reichenbachiella, preferably Prevotella, even more preferably from Prevotella sp. P5-125. In another embodiment, the Cas13b protein is, or comprises an amino acid sequence having at least 80% sequence identity to any of the sequences listed in Table 6 of US20200165594, which is herein incorporated by reference. In certain embodiments, the Cas13c protein is one as disclosed in U.S. Provisional Patent Application No. 62/525,165 filed Jun. 26, 2017, and PCT Application No. US 2017/047193 filed Aug. 16, 2017.

In a preferred embodiment of the present invention the Cas 13 protein encoded by the at least one nucleotide sequence is Cas13d or a functional variant thereof or a homologue or an orthologue thereof. Cas13d protein may be from an organism of a genus selected from the group consisting of Leptotrichia, Listeria, Corynebacter, Sutterella, Legionella, Treponema, Filifactor, Eubacterium, Ruminococcus, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira, preferably from Eubacterium or Ruminococcus, most preferably from Ruminococcus. It has been shown that Cas13d from Ruminococcus flavefaciens, preferably from Ruminococcus flavefaciens XPD3002, is most efficient to degrade viral target RNAs. Thus, in a preferred embodiment, the Cas13d protein encoded by the at least one nucleotide sequence is derived from Ruminococcus, even more preferably derived from Ruminococcus flavefaciens, most preferably Ruminococcus flavefaciens XPD3002. By derived, it is meant that the derived protein is largely based, in the sense of having a high degree of sequence homology with a wildtype protein, here with Cas13d from Ruminococcus, preferably from Ruminococcus flavefaciens, and thus provides the same function as said wildtype protein, but that it has been mutated (modified) in some way as known in the art or as described herein. A Cas13d protein that is derived from Ruminococcus, preferably from Ruminococcus flavefaciens, most preferably from Ruminococcus flavefaciens XPD3002 may also refer to a functional variant as defined elsewhere herein. A system as defined herein, wherein said at least one nucleotide sequence encoding said at least one Cas13 protein which is from the genus of Ruminococcus, preferably from Ruminococcus flavefaciens, most preferably from Ruminococcus flavefaciens XPD3002, is also comprised herein and mostly preferred.

In particular embodiments, the functional variant of said Cas13d protein as defined elsewhere herein has a sequence homology or identity of at least about 50%, or at least about 60%, or at least about 70%, or at least about 80%, more preferably at least about 85%, even more preferably at least about 90%, such as for instance at least about 95%, at least about 96%, at least about 97%, at least about 98% or even at least about 99% amino acid sequence identity with the wildtype amino acid sequence of the Cas13d protein of Ruminococcus flavefaciens (SEQ ID NO. 1). Thus, the present invention also comprises the system as defined elsewhere herein, which comprises said at least one nucleotide sequence encoding said at least one Cas13 protein comprising an amino acid sequence with at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO.: 1.

A “functional variant” of a protein as used herein refers to a variant of such protein which retains at least partially the activity of that protein. Functional variants may include mutants (which may be insertion, deletion, or substitutions as known to a person skilled in the art), including polymorphs, etc. Advantageous embodiments can involve engineered or non-naturally occurring Cas13 proteins as defined herein. Thus, a functional variant of a Cas13 protein as described herein retains the biological activity of the Cas13 protein from which it is derived. Generally, a Cas13 protein variant has at least about 50%, 60%, 70%, 75%, 80%, 85%, 90%, 95%, 96%, 97%, 98% or even at least about 99% amino acid sequence identity with the Cas 13 protein from which it is derived.

The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related. In particular embodiments, the homologue or orthologue of a Cas13 protein such as Cas13a, b, c or d as referred to herein has a sequence homology or identity of at least 80%, more preferably at least 85%, even more preferably at least 90%, such as for instance at least 95% with a Cas13 protein such as Cas13a, b, c or d. In further embodiments, the homologue or orthologue of a Cas13 protein such as Cas13a, b, c or d as referred to herein has a sequence identity of at least 80%, more preferably at least 85%, even more preferably at least 90%, such as for instance at least 95% with the wild type Cas13 protein such as Cas13a, b, c or d. Each embodiment which refers to the Cas13 protein as defined herein may also be applicable to the functional variant thereof or a homologue or an orthologue thereof.

By “identity” or “sequence identity” is meant a property of sequences that measures their similarity or relationship. The term “sequence identity” or “identity” as used in the present invention means the percentage of pair-wise identical residues—following (homology) alignment of a sequence of a polypeptide of the invention with a sequence in question—with respect to the number of residues in the longer of these two sequences. Identity is measured by dividing the number of identical residues by the total number of residues and multiplying the product by 100. The percentage of sequence identity can, for example, be determined herein using the program BLASTP, version blastp 2.2.5 (Nov. 16, 2002; cf. Altschul, S. F. et al. (1997) Nucl. Acids Res. 25, 3389-3402). In this embodiment the percentage of homology is based on the alignment of the entire polypeptide sequences (matrix: BLOSUM 62; gap costs: 11.1; cutoff value set to 10-3) including the respective sequences. It is calculated as the percentage of numbers of “positives” (homologous amino acids) indicated as result in the BLASTP program output divided by the total number of amino acids selected by the program for the alignment.

The novel systems as defined elsewhere herein may also comprise at least one nucleic acid molecule comprising at least one nucleotide sequence encoding said at least one Cas13 protein as defined herein. A nucleic acid molecule comprising a nucleotide sequence, or a nucleotide sequence encoding said Cas13 protein of said systems; and a nucleic acid molecule comprising a nucleotide sequence, or a nucleotide sequence encoding said gRNA include DNA, such as cDNA or genomic DNA, and RNA. Preferably, embodiments reciting “RNA” are directed to mRNA. In general, throughout the present invention the term “nucleotide sequence” can also be replaced by “nucleic acid molecule comprising a nucleotide sequence”. Thus, the present invention comprises said novel CRISPR system inter alia comprising at least one DNA sequence encoding said at least one Cas13 protein as defined herein and at least one DNA sequence encoding said at least one gRNA as defined herein, which may refer to the DNA-based system. The present invention however also comprises said novel CRISPR system inter alia comprising at least one RNA sequence (mRNA sequence) encoding said at least one Cas13 protein as defined herein and at least one gRNA as defined herein, which may refer to the RNA-based system.

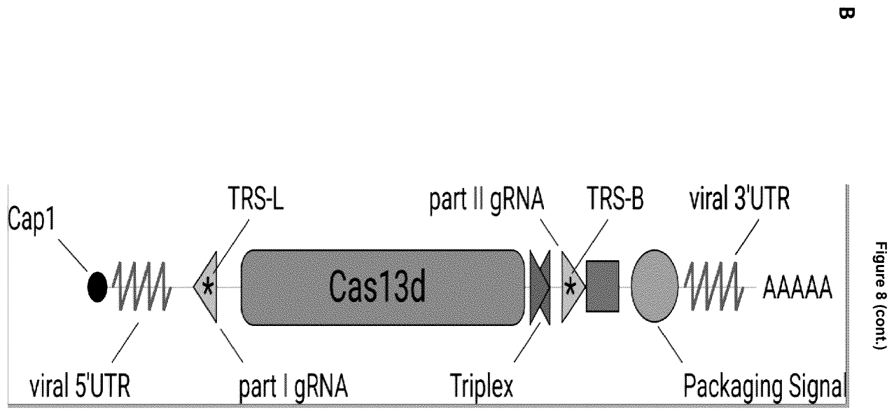

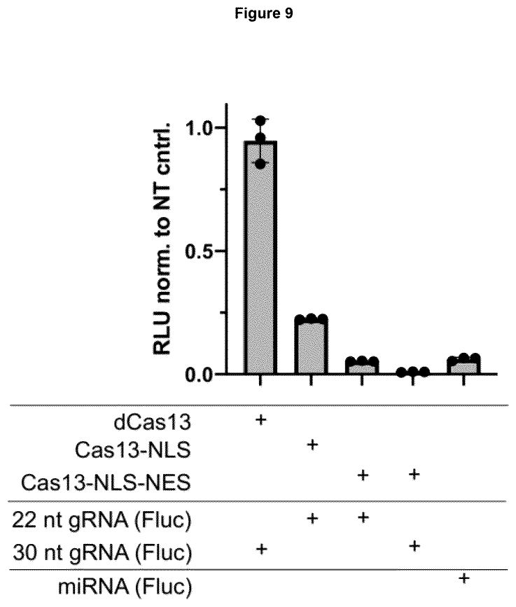

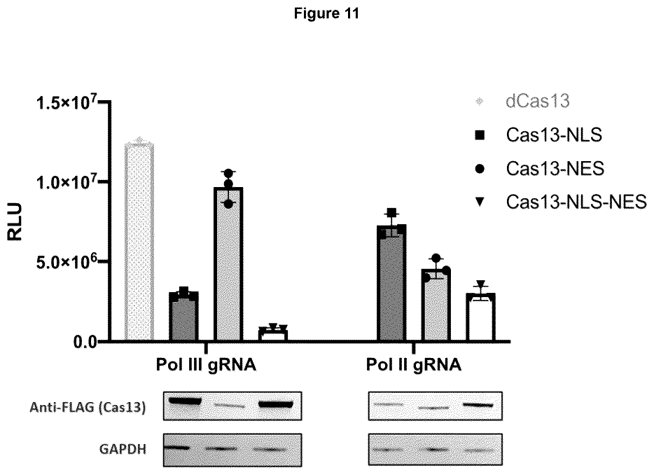

Additionally, the novel systems as defined elsewhere herein can be even more improved by three additional characteristics on the DNA or RNA level that increase the knockdown efficiency of the Cas13 protein and thus reduce the viral load. The first optimization step involves the fusion of at least one nucleotide sequence encoding at least one nuclear localization signal (NLS), which is fused to a nucleotide sequence encoding at least one nuclear export signal (NES), to the at least one nucleotide sequence encoding said at least one Cas13 protein. The fusion of at least one nucleotide sequence encoding at least one NLS fused to a nucleotide sequence encoding at least one NES to said nucleotide sequence encoding said Cas13 protein as defined herein has proven to be the best strategy for directing translated Cas13 protein into the nucleus, loading it with gRNA and then exporting it into the cytosol or vice versa, thereby leading to an increase in efficiency in the degradation of the desired target structures (see FIGS. 9 and 11). Thus, In a preferred embodiment, the novel CRISPR systems additionally use an inventive tandem localization signal of NLS and NES, whose encoding sequence is fused to the at least one nucleotide sequence encoding said at least one Cas13 protein as defined herein.

In general, gRNAs are transcribed in the cell nucleus, and according to the inventors, in order to activate the Cas13d RNAse activity, it is first necessary to bind the gRNA. Ideally, this step can only take place in the cell nucleus. In contrast, the viral RNA target molecules are essentially located in the cytosol after viral infection. The fusion of said localization signal allows the Cas13 protein to shuttle between the nucleus, where it may get activated by binding to said at least one gRNA, and the cytosol, where the target RNA molecules may be located for example after a viral infection of a subject. This allows degrading cytosolic instead of nuclear RNAs, which are important in the propagation of RNA viruses, such as coronaviruses, for which the replication takes place exclusively in the cytosol. However, if the systems of the present invention are not delivered as a DNA based system as mentioned above, but as an RNA based system, said systems comprising the above mentioned modification are also of high importance, e.g. for viruses that are located both in the nucleus and in the cytosol, such as for the family of Orthomyxoviridae, preferably for influenza viruses, in which case again a balanced localization of Cas13 protein is required in both cell compartments, which is achieved by the fusion of the at least one nucleotide sequence encoding at least one Cas13 protein to at least one nucleotide sequence encoding at least one NLS fused to at least one nucleotide sequence encoding at least one NES.

An NES is a short target peptide containing 4 hydrophobic residues in a protein that targets said protein, namely Cas 13 for export from the cell nucleus to the cytoplasm through the nuclear pore complex using nuclear transport, when for example the system is delivered as a DNA based system. An NLS targets a protein, namely Cas13, located in the cytoplasm for import to the nucleus, when for example the system is delivered as an RNA based system as defined elsewhere herein.

In another preferred embodiment, said at least one nucleotide sequence encoding said at least one Cas13 protein is fused with at least one nucleotide sequence encoding said at least one NLS fused to at least one nucleotide sequence encoding said at least one NES via a nucleotide sequence encoding a peptide linker known to a person skilled in the art of at least 1 amino acid in length. Thus, in this context, the wording implying that at least one nucleotide sequence encoding said at least one Cas13 protein as defined herein is linked to at least one nucleotide sequence encoding said at least one NLS fused to at least one nucleotide sequence encoding said at least one NES may also be used herein. This may also refer to an indirect fusion or linkage, since a peptide linker is applied. Thus, the wording “indirectly fused” or “indirectly linked” can also be used in this context. Said linker is preferably 1 to 20 amino acids in length. More preferably, the linker is 1 to 15 amino acids in length, and even more preferably, the linker is 1 to 10 amino acids in length, such as 1 to 5 amino acids in length. Even more preferably, the linker is 10, 9, 8, 7, 6, 5, 4, 3, 2 or 1 amino acid(s) in length. It is preferred that the linker molecule is a linear or a helical linker, even more preferably the linker is a helical linker. It is further preferred that the linker is a flexible linker, using e.g. the amino acids glycine and/or serine. In a particularly preferred embodiment of the invention, between 50% and 100%, particularly between 60% and 100%, particularly between 70% and 100%, particularly between 80% and 100%, particularly between 90% and 100%, and especially 100% of the amino acid residues of the linker molecule are glycine and serine residues, preferably forming an alpha-helix structure.

The fusion/linkage between the nucleotide sequence encoding Cas13 protein and the sequence encoding the localization signal as defined herein preferably includes a nucleotide sequence encoding a GlySer linker such as a GGS linker. They can be used in repeats of 3 ((GGS)3) or 6, 9 or even 12 or more, to provide suitable lengths, as required. The linkage may be covalent. In one embodiment, it may be comprised that said at least one nucleotide sequence encoding said at least one Cas13 protein is covalently fused/linked with said at least one nucleotide sequence encoding an NLS fused to said at least one nucleotide sequence encoding an NES as defined herein. The term “covalently linked/fused” in this context and also as used throughout the present invention refers to covalent bonds that are typically formed by the sharing of electron pairs between atoms. In accordance with the present invention and when the term “covalently fused/linked” is used, a covalent bond is formed between said Cas13 protein and said localization signal as defined herein by use of a peptide linker as described above, or such covalent bond is formed between said at least one NLS (also among each other, if more than one NLS is encoded) and said at least one NES (also among each other, if more than one NES is encoded) as defined elsewhere herein.

Said localization signal comprising at least one nucleotide sequence encoding at least one NLS fused to said at least one nucleotide sequence encoding said at least one NES as defined elsewhere herein may be fused to the 5′ or 3′ end of the at least one nucleotide sequence encoding said at least one Cas13 protein. Thus, in one embodiment, said localization signal comprising said at least one nucleotide sequence encoding said at least one NLS fused to said at least one nucleotide sequence encoding said at least one NES as defined elsewhere herein is fused to the 5′ end of the at least one nucleotide sequence encoding said at least one Cas13 protein. In another embodiment, said localization signal comprising said at least one nucleotide sequence encoding said at least one NLS fused to said at least one nucleotide sequence encoding said at least one NES as defined elsewhere herein is fused to the 3′ end of the at least one nucleotide sequence encoding said at least one Cas13 protein. According to another embodiment of the present invention, said at least one nucleotide sequence encoding said at least one NLS may be fused directly to said at least one nucleotide sequence encoding said at least one NES, meaning that the nucleotide sequences are arranged one after the other without a nucleotide sequence encoding a linker as defined elsewhere herein. A “direct fusion” or “fusing directly” as used throughout the present invention thus refers to fusing said at least one nucleotide sequence encoding said at least one NLS to said at least one nucleotide sequence encoding said at least one NES as defined elsewhere herein without any linker sequence. The definition of a direct fusion as above can be applied in any context concerning a direct fusion. The direct fusion or “fusing directly” also refers to the fusion of said nucleotide sequence encoding said NLS and/or said nucleotide sequence encoding said NES among each other, if more than one NLS or NES is used for said localization signal.

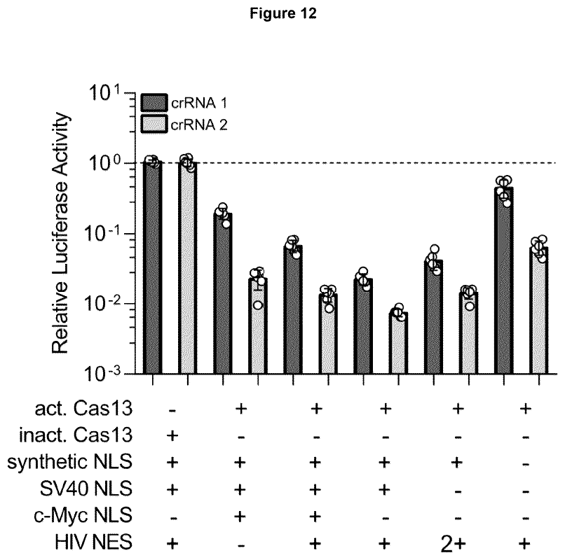

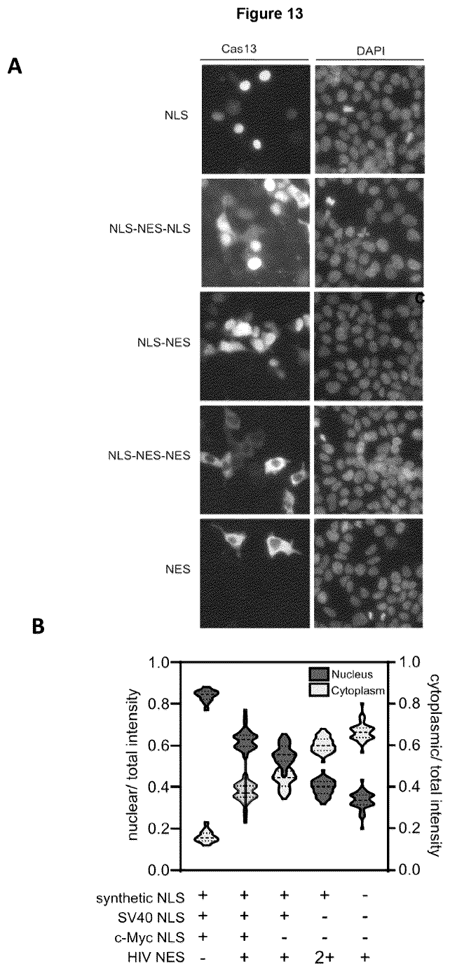

In this context, any arrangement, no matter if said at least one nucleotide sequence encoding said at least one Cas13 protein is firstly fused to said at least one nucleotide sequence encoding said NLS or firstly fused to said at least one nucleotide sequence encoding said NES, may be comprised herein. Thus, said at least one nucleotide sequence encoding said at least one Cas13 protein as defined herein may be fused/linked as described firstly to at least one nucleotide sequence encoding said at least one NLS followed by at least one nucleotide sequence encoding said at least one NES or vice versa. Also when one or more NLS and one or more NES are used for said localization signal, any arrangement within said localization signal is possible (e.g. NLS-NES-NLS or NES-NES-NLS or NLS-NLS-NES-NES etc.) and is thus comprised by the present invention. In another preferred embodiment, the novel systems as defined herein comprise that said at least one nucleotide sequence encoding said at least one Cas13 protein is fused with two nucleotide sequences both encoding an NLS fused to one nucleotide sequence encoding an NES (NLS-NES-NLS). Non-limiting examples of NLSs include, but are not limited to an NLS sequence derived from the NLS of the SV40 virus large T-antigen having the amino acid sequence PKKKRKV (SEQ ID NO: 2), an NLS-sequence derived from the extended version of said NLS of the SV40 virus large T-antigen having the amino acid sequence PPKKKRKVED (SEQ ID NO: 5), an NLS consensus sequence (also called synthetic NLS) having the amino acid sequence PAAKKKLD (SEQ ID NO: 3), a c-myc NLS having the amino acid sequence PAAKRVKLD (SEQ ID NO: 6), or a nucleoplasmin bipartite NLS with the amino acid sequence KRPAATKKAGQAKKKK (SEQ ID NO: 7). Non-limiting examples of NESs include, but are not limited to an NES sequence derived from the NES of the HIV virus having the amino acid sequence LQLPPLERLTL (SEQ ID NO: 4). In another preferred embodiment, the novel systems as defined herein comprise that the two NLS encoded by said two nucleotide sequences as defined above comprise the SV40 NLS having the amino acid sequence as depicted in SEQ ID NO: 5 and the NLS consensus sequence having the amino acid sequence as depicted in SEQ ID NO: 3 and the one NES encoded by said one nucleotide sequence comprises the HIV NES having the amino acid sequence as depicted in SEQ ID NO: 4 (see FIGS. 12 and 13). In this regard, any arrangement of said NLS according to SEQ ID NO: 5 and of said NLS according to SEQ ID NO. 3 and of said NES according to SEQ ID NO: 4 as defined is comprised herein by said novel systems. Preferably, said one nucleotide sequence encoding said SV40 NLS having the amino acid sequence as depicted in SEQ ID NO: 5 is fused/linked to the nucleotide sequence encoding said Cas13 protein and followed by/fused to said nucleotide sequence encoding said HIV NES having the amino acid sequence as depicted in SEQ ID NO: 4, which is then followed by/linked to said nucleotide sequence encoding said NLS consensus sequence (also called synthetic NLS) having the amino acid sequence as depicted in SEQ ID NO: 3.

The nucleotide sequence encoding said Cas13 protein, in particular Cas13d, is advantageously a codon optimized CRISPR protein. An example of a codon optimized sequence, is in this instance a sequence optimized for expression in eukaryotes, e.g., humans (i.e. being optimized for expression in humans), or for another eukaryote, animal or mammal as herein discussed. Whilst this is preferred, it will be appreciated that other examples are possible and codon optimization for a host species other than human, or for codon optimization for specific organs is known. In some embodiments, a nucleotide sequence encoding said Cas13 protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In general, codon optimization refers to a process of modifying a nucleotide sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. Codon usage tables are readily available, for example, at the “Codon Usage Database” available at kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding said Cas13 protein correspond to the most frequently used codon for a particular amino acid.

It will be readily appreciated by the skilled person that more than one nucleotide sequence may encode a Cas13 protein, preferably a Cas13d protein in accordance with the present invention due to the degeneracy of the genetic code. Degeneracy results because a triplet code designates 20 amino acids and a stop codon. Because four bases exist which are utilized to encode genetic information, triplet codons are required to produce at least 21 different codes. The possible 43 possibilities for bases in triplets give 64 possible codons, meaning that some degeneracy must exist. As a result, some amino acids are encoded by more than one triplet, i.e. by up to six. The degeneracy mostly arises from alterations in the third position in a triplet. This means that nucleotide sequences having different sequences, but still encoding the same Cas13 protein, can be employed in accordance with the present invention. The nucleotide sequences used in accordance with the present invention may be of natural as well as of (semi) synthetic origin. Thus, the nucleotide sequences may, for example, be nucleotide sequences that have been synthesised according to conventional protocols of organic chemistry. The person skilled in the art is familiar with the preparation and the use of said probes (see e.g. Sambrook and Russel “Molecular Cloning, A Laboratory Manual”, Cold Spring Harbor Laboratory, N.Y. (2001)). Also in accordance with the present invention, the nucleotide sequences used in accordance with the invention may be nucleic acid mimicking molecules known in the art such as synthetic or semi-synthetic derivatives of nucleotide sequences and mixed polymers. They may contain additional non-natural or derivatised nucleotide bases, as will be readily appreciated by those skilled in the art. Nucleic acid mimicking molecules or derivatives according to the invention include, without being limiting, phosphorothioate nucleic acid, phosphoramidate nucleic acid, morpholino nucleic acid, hexitol nucleic acid (HNA), peptide nucleic acid (PNA) and locked nucleic acid (LNA).

When the nucleotide sequence encodes as CRISPR protein a Cas13 protein, as it is the case in the present invention, a tracrRNA is not required in the composition as described elsewhere herein. However, said system also additionally comprises at least one guide RNA (gRNA) or at least one nucleotide sequence encoding said at least one gRNA capable of hybridizing with one or more viral target RNA molecules as defined herein. In this context, the novel system as defined elsewhere herein may also comprise on the DNA level at least one nucleic acid molecule comprising at least one nucleotide sequence encoding said at least one gRNA as defined herein. The definitions and preferred embodiments recited above with regard to the nucleotide sequence/nucleic acid molecule comprising at least one nucleotide sequence encoding said at least one Cas13 protein apply mutatis mutandis also to the nucleotide sequence/nucleic acid molecule comprising at least one nucleotide sequence encoding said at least one gRNA.

As used herein, the term “guide RNA”, “gRNA”, or “complementary crRNA” refers to a polynucleotide comprising any polynucleotide sequence having sufficient complementarity with one or more viral target RNA molecules to hybridize with the one or more viral target RNA molecules and to direct sequence-specific binding of an RNA-targeting complex comprising the gRNA and a CRISPR Cas13 protein to the one or more viral target RNA molecule. In general, a gRNA may be any polynucleotide sequence (i) being able to form a complex with a CRISPR protein such as Cas13 and (ii) comprising a sequence having sufficient complementarity with a target polynucleotide sequence to hybridize with the target sequence and direct sequence-specific binding of a CRISPR complex to the target sequence. As used herein the term “gRNA capable of hybridizing” refers to a subsection of the gRNA having sufficient complementarity to the one or more viral target RNA molecules to hybridize thereto and to mediate binding of a CRISPR complex to the target RNA. In general, a guide sequence, which is part of the gRNA, is any polynucleotide sequence having sufficient complementarity with a target polynucleotide sequence as defined elsewhere herein to hybridize with the target sequence and direct sequence-specific binding of a CRISPR complex to the target sequence. Concerning the present invention the selection of said any polynucleotide sequence having sufficient complementarity with a target polynucleotide sequence is performed by selection of any one target sequence within a corresponding viral genome infecting the respective subject, which presents a suitable target characterized in that its cutting from the infecting viral genome leads to the viral genome being rendered no longer functional for further propagation of and/or infection by the respective infecting virus. The identification of said suitable target sequences is within the scope of general practice in the art and well known to the skilled person such as identifying the primary sequence from a database or via sequencing and then generating the reverse complementary sequence hereto and can be supported by a wide range of tools assisting such identification, such as algorithms for identifying target sequences and/or designing gRNA sequences suited for a wide range of targets and implementations. In some examples, a plurality of gRNAs are generated from a single array, wherein each gRNA can be different, for example target different RNAs or target multiple regions of a single RNA, or combinations thereof. In a preferred embodiment, said gRNA being used herein by the systems of the invention refers to the gRNA having the nucleotide sequence as depicted in SEQ ID NO: 10.

As used herein the term “capable of forming a complex with the Cas 13 protein” refers to the gRNA having a structure that allows specific binding by the Cas 13 protein to the gRNA such that a complex is formed that is capable of binding to a viral target RNA molecule in a sequence specific manner and that can exert a function on said target RNA. Structural components of the gRNA may include direct repeats and a guide sequence (or spacer). The sequence specific binding to the viral target RNA is mediated by a part of the gRNA, the “guide sequence”, being complementary to the viral target RNA.

As mentioned above, said novel systems can be even more improved by three additional characteristics on the DNA or RNA level. Additionally or alternatively to the first optimization step as defined elsewhere herein, the second optimization step involves the optimization of the at least one gRNA or of the at least on nucleotide sequence encoding said at least one gRNA. Complementary sequences of 22 bases are usually used as standard. The at least one gRNA or the nucleotide sequence encoding said at least one gRNA as used herein and comprised by the systems of the present invention preferably comprise a length of at least about 23, such as at least about 24, at least about 25, at least about 26, at least about 27, at least about 28, at least about 29, or at least about 30 nucleotides. Thus, the gRNAs of the systems of the present invention can be optimized by extending their length as an additional modification to the novel systems as disclosed herein.

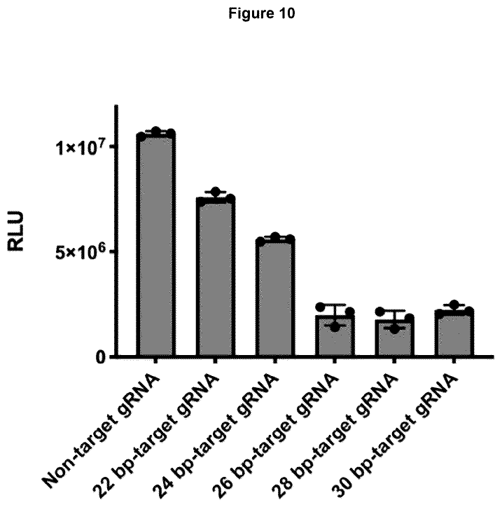

Said at least one gRNA or the nucleotide sequence encoding said gRNA comprised by the systems of the present invention comprises in a more preferred embodiment a length of between about 23 to about 30 nucleotides such as 23, 24, 25, 26, 27, 28, 29, or 30 nucleotides, preferably between about 24 to about 30 nucleotides, more preferably between about 25 to about 30 nucleotides, most preferably between about 26 to about 30 nucleotides. The inventors were able to show in particular that longer gRNAs comprising a length of between about 26 to about 30, between about 27 to about 30, between about 28 to about 30, between about 29 to about 30 nucleotides significantly increase the enzymatic activity of said Cas13 protein (see FIGS. 9 and 10). Preferably, the at least one gRNA has a length of about 26, 27, 28, 29, or about 30 nucleotides.

In a preferred embodiment, said novel CRISPR systems of the present invention comprising i) said at least one nucleotide sequence encoding said at least one Cas13 protein and ii) at least one gRNA or at least one nucleotide sequence encoding said at least one gRNA as defined herein, wherein said gRNA has a length of between about 26 to about 30 nucleotides is thus also envisaged herein. In another preferred embodiment, said novel CRISPR systems of the present invention comprising i) said at least one nucleotide sequence encoding said at least one Cas13 protein and ii) at least one gRNA or at least one nucleotide sequence encoding said at least one gRNA as defined herein, wherein said gRNA has a length of between about 27 to about 30 nucleotides is thus also envisaged herein. In another preferred embodiment, said novel CRISPR systems of the present invention comprising i) said at least one nucleotide sequence encoding said at least one Cas13 protein and ii) at least one gRNA or at least one nucleotide sequence encoding said at least one gRNA as defined herein, wherein said gRNA has a length of between about 28 to about 30 nucleotides is thus also envisaged herein. In another preferred embodiment, said novel CRISPR systems of the present invention comprising i) said at least one nucleotide sequence encoding said at least one Cas13 protein and ii) at least one gRNA or at least one nucleotide sequence encoding said at least one gRNA as defined herein, wherein said gRNA has a length of between about 29 to about 30 nucleotides is thus also envisaged herein. In an even more preferred embodiment, said novel CRISPR systems of the present invention comprising i) said at least one nucleotide sequence encoding said at least one Cas13 protein and ii) at least one gRNA or at least one nucleotide sequence encoding said at least one gRNA as defined herein, wherein said gRNA has a length of about 26 nucleotides is thus also envisaged herein. In another even more preferred embodiment, said novel CRISPR system of the present invention comprising i) said at least one nucleotide sequence encoding said at least one Cas13 protein and ii) at least one gRNA or at least one nucleotide sequence encoding said at least one gRNA as defined herein, wherein said gRNA has a length of about 27 nucleotides is thus also envisaged herein. In another even more preferred embodiment, said novel CRISPR system of the present invention comprising i) said at least one nucleotide sequence encoding said at least one Cas13 protein and ii) at least one gRNA or at least one nucleotide sequence encoding said at least one gRNA as defined herein, wherein said gRNA has a length of about 28 nucleotides is thus also envisaged herein. In another even more preferred embodiment, said novel CRISPR system of the present invention comprising i) said at least one nucleotide sequence encoding said at least one Cas13 protein and ii) at least one gRNA or at least one nucleotide sequence encoding said at least one gRNA as defined herein, wherein said gRNA has a length of about 29 nucleotides is thus also envisaged herein. In another even more preferred embodiment, said novel CRISPR system of the present invention comprising i) said at least one nucleotide sequence encoding said at least one Cas13 protein and ii) at least one gRNA or at least one nucleotide sequence encoding said at least one gRNA as defined herein, wherein said gRNA has a length of about 30 nucleotides is thus also envisaged herein. Such embodiments may also be combined with the first optimization step of said NLS-NES fusion as defined elsewhere herein.

In a preferred embodiment said at least one gRNA or the at least one nucleotide sequence encoding said at least one gRNA has at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 85%, at least about 90%, at least about 95%, at least about 96%, at least about 97%, at least about 98%, at least about 99% or even 100% complementary sequence identity to the one or more viral target RNA molecules. The inventors found out that having one mismatch with the one or more target RNA molecules could be well tolerated, which would refer, depending on the length of the gRNA, to at least about 95% complementary sequence identity to the one or more viral target RNA molecules. If the distal end comprising 7 nucleotides is modified (mismatches occur so that less than 80% complementary sequence identity to the one or more viral target RNA molecules are present), the gRNA having a length of about 30 nucleotides would still be functional. Thus, mismatches on the distal end may be more tolerated than on the proximal end of said gRNA. Preferably, said at least one gRNA or the at least one nucleotide sequence encoding said at least one gRNA has at least about 70% complementary sequence identity to the one or more target RNA molecules. In some embodiments, the degree of complementarity between a guide sequence as part of the gRNA as defined elsewhere herein and its corresponding viral target sequence comprised by the viral target RNA molecule, when optimally aligned using a suitable alignment algorithm, is about or more than about 68%, 70%, 75%, 80% 85%, 90%, 95%, 96%, 97%, 98%, 99% or more, preferably about or more than about 70%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at novocraft.com), ELAND (Illumina, San Diego, Calif.), SOAP (available at soap.genomics.org.cn), and Maq (available at maq. sourceforge.net).

At least one (one or more), such as one, two, three, four, five and more gRNAs or at least one nucleotide sequence encoding said at least one gRNA may be used in the novel systems according to the present invention. Thereby, each of the gRNAs can bind to a viral sequence in the genome. The simultaneous application of more than one gRNA or more than one nucleotide sequence encoding said one or more gRNA that address several regions of the virus genome may also be applied in terms of the present invention. This refers to a process called “multiplexing”. Thus, even if the virus mutates, efficient and sustained degradation can be ensured.

In certain embodiments, the at least one (one or more) gRNA is capable of binding/binds to the coding strand of the RNA. In certain embodiments, the gRNA is capable of binding/binds to the non-coding strand of the RNA. In certain embodiments, the gRNA binds to viral genomic RNA (positive (+) or negative (−) sense or antisense strand). In certain embodiments, the gRNA binds to transcribed RNA (positive or negative sense or coding or non-coding strand) from viral genomic DNA.

In a preferred embodiment, said at least one (one or more) gRNA is capable of hybridizing/binding or hybridizes/binds to a 5′- and/or 3′ untranslated region(s) of said one or more viral target RNA molecules. The term “non-coding region”/“non-coding sequence” may be used interchangeably with the term “untranslated region”. Since these two areas are present both in the genome of any virus and in all sub-genomic mRNAs, the antiviral effect of Cas13 is strongest at these positions.

Additionally or alternatively to the first and/or second optimization step as defined elsewhere herein, the third optimization step on the DNA level involves the fusion of the nucleotide sequence encoding said gRNA with a nucleotide sequence encoding a tRNA which proves to be advantageous, since it supports the folding of the gRNA. Once the system comprising such optimization has been administered to a subject, the nucleotide sequence encoding said gRNA fused with the nucleotide sequence encoding the tRNA is transcribed into a single RNA molecule in which the gRNA is fused as RNA to the tRNA as RNA. The same applies to the ribozyme as defined below. Said nucleotide sequence encoding said tRNA may be fused directly as defined elsewhere herein to the 3′ end of the nucleotide sequence encoding said gRNA, wherein said nucleotide sequence is DNA. Thus, the system of the present invention is also comprised herein, wherein the DNA nucleotide sequence encoding said gRNA is fused directly with a nucleotide sequence encoding a tRNA at the 3′ end of said nucleotide sequence. Any tRNA known to a person skilled in the art may be used. The predominance of selected tRNAs in a cell is generally a reflection of the codons used most frequently in peptide synthesis. Accordingly, genes can be tailored for optimal gene expression in a given organism based on codon optimization as defined elsewhere herein. Preferably, the tRNA from or derived from murid y-herpesvirus 68 (MHV68) having the nucleotide sequence as depicted in SEQ ID NO: 8 is used for said fusion. In this context and also with regard to the fusion with a ribozyme, the definition of “derived” as defined elsewhere herein may be applied, also having at least about 50%, 60%, 70%, 75%, 80%, 85%, 90%, 95%, 96%, 97%, 98% or even at least about 99% sequence identity with the nucleotide sequence encoding said tRNA or said ribozyme from which it is derived. It is also comprised by the present invention that said nucleotide sequence encoding said RNA may be fused to said nucleotide sequence encoding said gRNA, preferably to the 3′ end of the nucleotide sequence encoding said gRNA, as defined elsewhere herein via a linker as defined herein.

Alternatively, said nucleotide sequence encoding said gRNA, wherein said sequence refers to a DNA sequence, can also be fused directly as defined herein as another optimization step to a nucleotide sequence encoding a ribonucleic acid enzyme (short: ribozyme). A “ribozyme” is an RNA molecule that has the ability to catalyze specific biochemical reactions, including RNA splicing in gene expression as known to a person skilled in the art. A natural or even a synthetic ribozyme may be used herein for said fusion. In a preferred embodiment, said ribozyme is or is derived as defined elsewhere herein from Hepatitis delta virus (HDV) having the nucleotide sequence as depicted in SEQ ID NO: 9. Contrary to the tRNA being used and defined herein, which may be split off by RNAse P and RNAse Z after a certain period of time, which results in a clean defined terminal end of said gRNA, preferably in a clean defined 3′-terminal end of said gRNA, the ribozyme being used herein excises itself from said gRNA, however also resulting in a clean defined terminal end of said gRNA, preferably in a clean defined 3′terminal end of said gRNA—both modifications then leading to an efficiency improvement as described herein.

Also comprised herein by the term “ribozyme” are isolated ribozymes or ribozyme portions being encoded by a nucleotide sequence which are fused to said nucleotide sequence encoding said gRNA. Such ribozymes comprise a motif which specifically covalently fuses with said sequence, with the result that the ribozyme is joined specifically to the sequence. Ribozymes used herein can be a contiguous sequence or can be comprised of two noncontiguous components which interact with one another to form the complete ribozyme. The two noncontiguous components are a ribozyme segment which specifically covalently fuses to said nucleotide sequence and a ribozyme segment which comprises the remaining ribozyme. The remaining ribozyme sequence is the ribozyme sequence which, in combination with the ribozyme segment which covalently fuses to said nucleotide sequence, makes up the complete ribozyme. The ribozyme sequence or segment which covalently fuses to said sequence can be as short as one nucleotide in length and can be from any location (e.g., 5′ end, internal segment, 3′ end) in the ribozyme. In one embodiment, the ribozyme segment includes from one to about 18 nucleotides, such as from the first to about the 18th nucleotide (from the 5′ end) of a ribozyme. In further embodiments, the ribozyme segment is the first 13 to 18 nucleotides (from the 5′ end) of the ribozyme (e.g., the first 13, 14, 15, 16, 17, or 18 nucleotides from the 5′ end). The other component is the remaining ribozyme sequence (the remainder of the ribozyme which is necessary to form a functional ribozyme). The two ribozyme components interact with one another to form a functional (complete) ribozyme. Preferably, said nucleotide sequence encoding said ribozyme as defined elsewhere herein is fused directly as defined elsewhere herein to the 3′ end of the nucleotide sequence encoding said gRNA, wherein said nucleotide sequence is DNA. It is also comprised by the present invention that said nucleotide sequence encoding said ribozyme as defined elsewhere may be fused to said nucleotide sequence encoding said gRNA, preferably to the 3′ end of the nucleotide sequence encoding said gRNA as defined herein via a linker as defined herein.

As mentioned above, the fusion of said nucleotide sequence encoding said tRNA and/or said ribozyme, which can be a direct fusion to the DNA nucleotide sequence encoding said gRNA, may be covalent. The present invention thus further comprises that said nucleotide sequence (DNA sequence) encoding said gRNA is (directly) fused covalently with a nucleotide sequence encoding said tRNA and/or said ribozyme. In this context, when the term “covalently fused” is used, a covalent bond is formed between the nucleotide sequence encoding said gRNA of the present invention and the nucleotide sequence encoding said tRNA or said ribozyme as defined above. In these embodiments, the term “directly” again means that the nucleotide sequences encoding said gRNA and the tRNA and/or ribozyme are arranged one after the other without a linker as defined elsewhere herein. Alternatively, the nucleotide sequences encoding said gRNA and the tRNA and/or ribozyme may also be fused via a linker as defined elsewhere herein.

In a preferred embodiment, said novel systems of the present invention comprising i) at least one nucleotide sequence encoding said at least one Cas13 protein and ii) at least one nucleotide sequence encoding said at least one gRNA as defined herein, wherein said gRNA has a length of between about 26 to about 30 nucleotides and wherein said nucleotide sequence encoding said gRNA is also fused to a nucleotide sequence encoding a tRNA or a ribozyme as defined is thus also envisaged herein. In another preferred embodiment, said novel systems of the present invention comprising i) at least one nucleotide sequence encoding said at least one Cas13 protein and ii) at least one nucleotide sequence encoding said at least one gRNA as defined herein, wherein said gRNA has a length of between about 27 to about 30 nucleotides and wherein said nucleotide sequence encoding said gRNA is also fused to nucleotide sequence encoding a tRNA or a ribozyme as defined is thus also envisaged herein. In another preferred embodiment, said novel systems of the present invention comprising i) at least one nucleotide sequence encoding said at least one Cas13 and ii) at least one nucleotide sequence encoding said at least one gRNA as defined herein, wherein said gRNA has a length of between about 28 to about 30 nucleotides and wherein said nucleotide sequence encoding said gRNA is also fused to a nucleotide sequence encoding a tRNA or a ribozyme as defined is thus also envisaged herein. In another preferred embodiment, said novel systems of the present invention comprising i) at least one nucleotide sequence encoding said at least one Cas13 protein and ii) at least one nucleotide sequence encoding said at least one gRNA as defined herein, wherein said gRNA has a length of between about 29 to about 30 nucleotides and wherein said nucleotide sequence encoding said gRNA is also fused to a nucleotide sequence encoding a tRNA or a ribozyme as defined is thus also envisaged herein. In an even more preferred embodiment, said novel systems of the present invention comprising i) at least one nucleotide sequence encoding said at least one Cas13 protein and ii) at least one nucleotide sequence encoding said at least one gRNA as defined herein, wherein said gRNA has a length of about 26 nucleotides and wherein said nucleotide sequence encoding said gRNA is also fused to a nucleotide sequence encoding a tRNA or a ribozyme as defined is thus also envisaged herein. In another even more preferred embodiment, said novel systems of the present invention comprising i) at least one nucleotide sequence encoding said at least one Cas13 protein and ii) at least one nucleotide sequence encoding said at least one gRNA as defined herein, wherein said gRNA has a length of about 27 nucleotides and wherein said nucleotide sequence encoding said gRNA is also fused to a nucleotide sequence encoding a tRNA or a ribozyme as defined is thus also envisaged herein. In another even more preferred embodiment, said novel systems of the present invention comprising i) at least one nucleotide sequence encoding said at least one Cas13 protein and ii) at least one nucleotide sequence encoding said at least one gRNA as defined herein, wherein said gRNA has a length of about 28 nucleotides and wherein said nucleotide sequence encoding said gRNA is also fused to a nucleotide sequence encoding a tRNA or a ribozyme as defined is thus also envisaged herein. In another even more preferred embodiment, said novel systems of the present invention comprising i) at least one nucleotide sequence encoding said at least one Cas13 protein and ii) at least one nucleotide sequence encoding said at least one gRNA as defined herein, wherein said gRNA has a length of about 29 nucleotides and wherein said nucleotide sequence encoding said gRNA is also fused to a nucleotide sequence encoding a tRNA or a ribozyme as defined is thus also envisaged herein. In another even more preferred embodiment, said novel systems of the present invention comprising i) at least one nucleotide sequence encoding said at least one Cas13 protein and ii) at least one nucleotide sequence encoding said at least one gRNA as defined herein, wherein said gRNA has a length of about 30 nucleotides and wherein said nucleotide sequence encoding said gRNA is also fused to a nucleotide sequence encoding a tRNA or a ribozyme as defined is thus also envisaged herein. Such embodiments may also be combined with the first optimization step of NLS-NES fusion as defined elsewhere herein.

In addition to the optimization steps as defined herein or alternatively, the gRNA can also be modified on the RNA level, preferably with regard to the system variant 3 as defined elsewhere herein, wherein said at least one gRNA is comprised in the second construct of said system variant 3 without further comprising said viral 5′ UTR and/or said viral 3′ UTR and thus not being replicated. The gRNA modification on the RNA level comprises the replacement of the (two) 5′ and/or 3′ terminal nucleotides by 2′-O-methyl-3′P-thioate (also called 2′-O-methyl phosphorothioate), which improves the knockdown-efficiency of the Cas13 protein even more, Thus, the present invention also refers to said novel systems as defined herein, wherein said at least one nucleotide sequence encoding said at least one Cas13 protein and said at least one gRNA are on two different nucleotide constructs of said system, the first construct comprising said at least one nucleotide sequence encoding said at least one Cas13 protein further comprises said viral 5′ UTR and/or said viral 3′ UTR, wherein said viral replicase recognition sequence is comprised in at least any one of said 5′ UTR or said 3′ UTR; and the second construct comprising said at least one gRNA, which is modified by replacing the (two) 5′ and/or 3′ terminal nucleotides of said gRNA by 2′-O-methyl-3′P-thioate. In this particular system variant said second construct comprising said at least one gRNA will not be replicated, so that the gRNA can be modified as mentioned above for stabilization. Such gRNA modification includes that the 2′OH on the sugar of the RNA may be replaced by a methyl group and that the phosphodiester bond in the RNA backbone may be modified by sulfur, whereby the gRNA becomes resistant to RNAses.

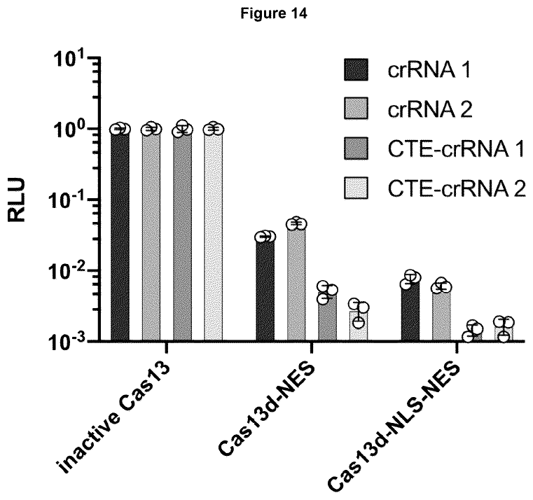

Further, the inventors have also found that another modification of the gRNA of said systems on the DNA level which comprises fusing at least one nucleotide sequence encoding at least one viral export element, preferably fusing at least one nucleotide sequence encoding at least one constitutive transport element (CTE) or at least one VARdm, most preferably fusing at least one nucleotide sequence encoding at least one CTE with said at least one nucleotide sequence encoding said at least one gRNA, increases the knockdown efficiency of the Cas13 protein for DNA delivery, which is fused—when encoded—to at least one NLS which is fused to at least one NES as defined above, even more (see FIG. 14). Such RNA export elements, such as CTE or VARdm, drive the export of gRNAs from the nucleus to the cytosol. Said at least one nucleotide sequence encoding at least one viral export element as defined herein is then fused as defined elsewhere herein to the 5′ end of the at least one nucleotide sequence encoding said at least one gRNA, preferably wherein said nucleotide sequence is DNA. In this context, fusing means that the nucleotide sequence encoding said gRNA and the at least one viral export element are fused via a linker, such as any nucleotide linker. Thus, said novel systems of the present invention are also comprised herein, wherein the at least one DNA nucleotide sequence encoding said at least one gRNA is fused via a linker such as any nucleotide linker with at least one nucleotide sequence encoding at least one viral export element as defined herein at the 5′ end of said at least one nucleotide sequence. The term “at least one viral export element” means that also two, three, four, five or more viral export elements as defined herein may be used. In a preferred embodiment, said at least one viral export element is a CTE or a VARdm, preferably a CTE, which is fused as defined herein with the nucleotide sequence encoding said gRNA, which is preferably a DNA sequence.

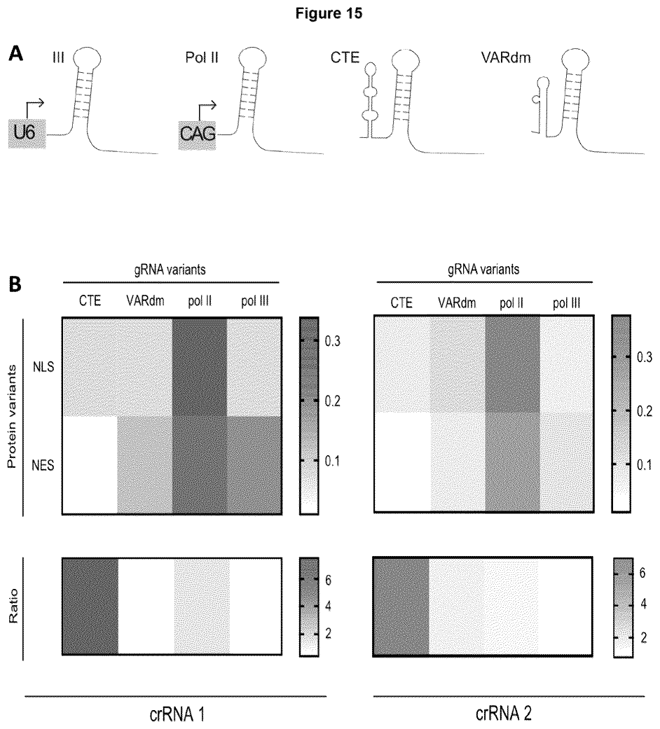

The inventors have also found out that the same effect, namely an increased efficiency in the degradation of the desired viral target structures, is achieved by said novel systems of the present invention comprising i) at least one nucleotide sequence encoding said at least one Cas13 protein fused with at least one nucleotide sequence encoding at least one NLS or with at least one nucleotide sequence encoding at least one NES; and ii) at least one nucleotide sequence encoding said at least one gRNA capable of hybridizing with one or more viral target RNA molecules, which is fused with at least one nucleotide sequence encoding at least one viral export element as defined elsewhere herein. Here, by fusing at least one nucleotide sequence encoding said at least one viral export element to said at least one nucleotide sequence encoding said at least one gRNA, cytosolic knockdown by Cas13-NES or NLS is increased (see FIG. 15). Said at least one nucleotide sequence encoding at least one viral export element as defined herein is fused as defined elsewhere herein to the 5′ end of the nucleotide sequence encoding said gRNA, preferably wherein said nucleotide sequence is DNA. In this context, fusing means again that the nucleotide sequence encoding said gRNA and at least one nucleotide sequence encoding at least one viral export element are fused via a linker such as any nucleotide linker. Again, the term “at least one viral export element” refers to two, three, four, five or more viral export elements as defined herein. In a preferred embodiment, said at least one viral export element is a constitutive transport element (CTE) or a minihelix of adenovirus VA1 RNA (VARdm), preferably a CTE, which is fused as defined herein with the nucleotide sequence encoding said gRNA, which is preferably a DNA sequence.

In some embodiments, the nucleotide sequences encoding said Cas13 protein and said gRNA may also be “operably linked” to one another within said systems, especially in system variant 1, i.e. as described above the nucleotide sequence encoding said at least one Cas13 protein as described elsewhere herein (if more than one Cas13 protein is used, also more than one nucleotide sequences encoding said Cas13 proteins may be applied), a nucleotide sequence encoding said at least one gRNA as defined elsewhere herein (if more than one gRNA is used, also more than one nucleotide sequences encoding said gRNAs may be applied), and a nucleotide sequence encoding said 5′ UTR and/or a nucleotide sequence encoding said 3′ UTR as defined elsewhere herein. In this regard, an operable linkage is a linkage in which the sequence elements of one nucleotide sequence and the sequence elements of another nucleotide sequence are connected in a way that enables expression of the system.

The gRNA(s) nucleotide sequences and/or Cas13 nucleotide sequences of said system variants can also be functionally or operatively linked to regulatory element(s). An operable linkage is a linkage in which the regulatory element(s) to be expressed are connected in a way that enables or modifies gene expression. Such regulatory elements may be derived from the same virus which the viral replicase recognition sequence is from. The precise nature of the regulatory elements necessary for gene expression may vary among the respective infecting virus, but in general these elements include a promoter which, in prokaryotes, contains both the promoter per se, i.e. DNA elements directing the initiation of transcription, as well as DNA elements which, when transcribed into RNA, will signal the initiation of translation. Those elements are well known to those skilled in the art and include, without being limiting, regulatory elements ensuring the initiation of transcription, termination of transcription and stabilization of the transcript, internal ribosomal entry sites (IRES) and/or transcriptional and/or translational termination (Owens, Proc. Natl. Acad. Sci. USA 98 (2001), 1471-1476). If, however, these termination sequences are not satisfactory functional in a particular virus infecting said subject, then they may be substituted with signals functional with regard to said virus. In a preferred embodiment, said regulatory elements additionally being comprised in said systems, refer to at least one nucleotide sequence encoding a translational termination sequence such as a Triplex sequence or an xrRNA. Such sequences may also have stabilization function for said systems of the present invention on the RNA level when also being comprised within to make sure the RNA constructs are longer functional and do not degrade. In an even more preferred embodiment, said sequence refers to a Triplex sequence. With comprising said translational termination sequence within said systems, the nucleotide sequences (preferably the mRNA sequences) of said systems are stabilized, meaning that for example if the gRNA is processed out off said systems, the remaining nucleotide sequence (preferably mRNA) encoding said at least one Cas13 protein of said systems may become instabil. By incorporating said translational termination sequence such as a Triplex sequence within said system which stabilizes the nucleotide sequences (preferably mRNA) encoding said at least one Cas13 protein, Cas13 protein can still be generated. With regard to the different system variants as defined elsewhere herein, said translational termination sequence may be comprised either in between said nucleotide sequence encoding said Cas13 protein and said gRNA or said nucleotide sequence encoding said gRNA (see system variant 1, FIG. 5; or system variant 4, FIG. 8) or, if two nucleotide constructs are applied for said system, said sequence may only be comprised on the construct comprising said nucleotide sequence encoding said Cas13 protein, in particular at the 3′ end of said nucleotide sequence encoding said Cas13 protein (see system variant 2, FIG. 6; or system variant 3, FIG. 7).

In another preferred embodiment, said novel systems as defined herein additionally comprise at least one viral packaging signal or at least one nucleotide sequence encoding said at least one viral packaging signal (see FIGS. 5B-8B). With regard to the system variant 1 or 4 (see FIGS. 5B and 8B), said at least one viral packaging signal or at least one nucleotide sequence encoding said at least one viral packaging signal may be comprised anywhere within said DNA or RNA construct. With regard to the system variant 2 (see FIG. 6B), said at least one viral packaging signal or at least one nucleotide sequence encoding said at least one viral packaging signal may be comprised anywhere within both DNA or RNA constructs (within said first construct comprising said nucleotide sequence encoding said Cas13 protein and within said second construct comprising said gRNA or said nucleotide sequence encoding said gRNA). With regard to the system variant 3 (see FIG. 7B), said at least one viral packaging signal or at least one nucleotide sequence encoding said at least one viral packaging signal may be comprised anywhere within said DNA or RNA construct which only comprises said nucleotide sequence encoding said Cas13 protein. A viral packaging signal refers to a viral recognition sequence for the packaging of said mRNA into viral capsid(s), thus enabling the secretion of the novel systems as it is also done so with the replicated viral RNA. Meaning, instead of only packaging viral RNA, said antiviral systems may additionally be packaged and then secreted. Thus, said novel systems may then be able to infect further cells within the subject as it is done naturally by said virus, which then refers to a co-distribution and a co-infection. Due to the co-distribution, co-infected cells may receive the antiviral systems along with the infecting virus. Replication of the actual virus is therefore then reduced (1) due to the simultaneous use of the viral replication machinery for Cas13 mRNA replication and (2) due to Cas13 activity rendering the viral genome without function. In sum, the application of said viral packaging signal within said novel systems, which preferably is from the same virus as defined herein as the viral replicase recognition sequence is from (which is the same virus which has infected the subject) may lead to packaging of the replicated mRNA so that it is secreted with actual virus particles, thus increasing distribution and efficiency. Only for system variant 3 (see FIG. 7B) the second construct comprising the gRNA or said nucleotide sequence encoding said gRNA is not packaged and thus not secreted. In non-infected cells absorbing Cas13-loaded capsids no replication or secretion may take place for all constructs, which causes the antiviral to be broken down. An overdose in non-infected cells may thus not take place because degradation of the antiviral system may occur, when no virus and thus no suitable viral replicase is present.

### Target RNA Molecules

As already mentioned herein, said at least one gRNA is capable of hybridizing with one or more viral target RNA molecules. In the context of formation of a CRISPR complex. “viral target sequence” refers to a viral sequence to which a guide sequence (part of the gRNA) is designed to have complementarity as defined elsewhere herein, where hybridization between a target sequence and a guide sequence promotes the formation of a CRISPR complex. A target sequence may comprise RNA polynucleotides. The term “viral target RNA molecules” refers to a viral RNA polynucleotide (target RNA) being or comprising the viral target sequence. In other words, the viral target RNA may be a viral RNA polynucleotide or a part of a RNA polynucleotide to which a part of the gRNA, i.e. the guide sequence, is designed to have complementarity as described herein and to which the effector function mediated by the complex comprising CRISPR effector protein and a gRNA is to be directed. In some embodiments, a viral target RNA molecule and thus a viral target RNA sequence is located in the nucleus and/or cytoplasm of a cell. The viral target RNA, i.e. the RNA of interest, is the RNA to be targeted by the present invention leading to the recruitment to, and the binding of the Cas13 protein at, the target site of interest on the viral target RNA. The one or more viral target RNA molecules may be any suitable form of RNA. This may include, in some embodiments, mRNA. In other embodiments, the one or more viral target RNA molecules may include tRNA or rRNA. In other embodiments, the one or more viral target RNA molecules may include miRNA. In other embodiments, the one or more viral target RNA molecules may include siRNA.

In some embodiments, the one or more viral target RNA molecules comprising the target sequence or also the target sequence per se may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non coding RNA (ncRNA), long non-coding RNA (IncRNA), and small cytoplasmatic RNA (scRNA). In some preferred embodiments, one or more viral target RNA molecules comprising the target sequence or also the target sequence per se may be a sequence within an RNA molecule selected from the group consisting of mRNA, pre-mRNA, and rRNA. In some other preferred embodiments, the one or more viral target RNA molecules comprising the target sequence or also the target sequence per se may be a sequence within an RNA molecule selected from the group consisting of ncRNA, and IncRNA. In some other preferred embodiments, the one or more viral target RNA molecules comprising the target sequence or also the target sequence per se may be a sequence within an mRNA molecule or a pre-mRNA molecule.

Thus, the at least one gRNA capable of hybridizing as defined elsewhere herein with one or more viral target RNA molecules may also comprise hybridization with one or more viral RNA target sequences. Preferably, by targeting the one or more viral target RNA molecules/RNA target sequences the at least one gRNA that is complementary to a certain extent as defined elsewhere herein to said molecules/target sequences focus on the 5′ and/or 3′ untranslated regions/parts of said RNA molecules (mRNA molecules) as already defined elsewhere herein.

In particular embodiments, the virus (the infecting virus) is an RNA virus. In further embodiments, the virus is a single stranded or double stranded RNA virus. In further embodiments, the virus is a positive sense RNA virus or a negative sense RNA virus or an ambisense RNA virus. In further embodiments, the virus is a Retroviridae virus, Lentiviridae virus, Coronaviridae virus, a Picornaviridae virus, a Caliciviridae virus, a Flaviviridae virus, a Togaviridae virus, a Bomaviridae, a Filoviridae, a Paramyxoviridae, a Pneumoviridae, a Rhabdoviridae, an Arenaviridae, a Bunyaviridae, an Orthomyxoviridae, or a Deltavirus. In particular embodiments, the virus is selected from the group consisting of Lymphocytic choriomeningitis virus, Coronavirus, HIV, SARS, Venezuelan Equine Encephalitis (VEE) virus, Poliovirus, Rhinovirus, Hepatitis A, Norwalk virus, Yellow fever virus, West Nile virus, Hepatitis C virus, Dengue fever virus, bunya virus, respiratory syncytial virus (RSV), Zika virus, Rubella virus, Ross River virus, Sindbis virus, Chikungunya virus, Boma disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza and Hepatitis D virus. In another particular embodiments, the virus is a DNA virus. In further embodiments, the virus is a single stranded or double stranded DNA virus. In further embodiments, the virus is a positive sense DNA virus or a negative sense DNA virus or an ambisense DNA virus. In further embodiments, the virus is a Myoviridae, Podoviridae, Siphoviridae, Alloherpesviridae, Herpesviridae (including human herpes virus, and Varicella Zozter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, or Rhizidovirus. In certain example embodiments, the virus may be a retrovirus. Example retroviruses may include one or more of or any combination of viruses of the Genus Alpharetrovirus, Betaretrovirus, Gammaretrovirus, Deltaretrovirus, Epsilonretrovirus, Lentivirus, Spumavirus, or the family Metaviridae, Pseudoviridae, and Retroviridae (including HIV), Hepadnaviridae (including Hepatitis B virus), and Caulimoviridae (including Cauliflower mosaic virus). In certain embodiments, the virus is a drug resistant virus and thus belongs to a multi-resistant germ as defined elsewhere herein. By means of example, and without limitation, the virus may be a ribavirin resistant virus. Ribavirin is a very effective antiviral that hits a number of RNA viruses. In a more preferred embodiment the term “viral” as used throughout the present invention refers to a coronavirus, influenza A virus, ebola virus, morbilivirus, hepacivirus, flavivirus such as TBE virus, venezuelan equine encephalitis virus, dengue virus, yellow fever virus or zika virus.

Thus, also comprised by the present invention is that said novel systems as defined herein are suitable for inactivating viral single-stranded (ss) RNA. In this context, the term “inactivating” or “inactivate” or “inactivation” refers to the systems as defined herein, wherein said at least one encoded Cas13 protein forms a complex with the at least one gRNA and wherein the at least one gRNA directs the complex to the one or more viral target RNA molecules, which not only targets/detects the one or more viral target RNA molecules, but then also cleaves/degrades said viral target RNA molecules. Thus, the term “cleaving”, “cleave”, “cleavage”, “cut”, “cutting” or “degrading”, “degrade” or “degradation” can also be used interchangeably with the term “inactivating”, “inactivate” or “inactivation”.

### Delivery System

The present invention also relates to a delivery system comprising said novel systems as defined herein. Such delivery systems as used herein which said systems of the present invention can be delivered with on a DNA level refer to any suitable vectors, e.g., a plasmid or a viral vector, such as an adeno associated virus (AAV), lentivirus, adenovirus (AV) vector or other viral vector types, or combinations thereof. As used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g. non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Such systems of the present invention can be packaged into one or more vectors, e.g., plasmids or viral vectors. In some embodiments, the vector, e.g., plasmids or viral vectors is delivered by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated as mentioned elsewhere herein, the degree of transformation/modification sought, the administration route, the administration mode, the type of transformation/modification sought, etc. Such a dosage may further contain a carrier as defined elsewhere herein. In one embodiment herein the delivery of said systems as defined herein based on DNA level is via an adenovirus or adeno associated virus, which may be at a single booster dose. In another embodiment herein, said systems as defined herein based on DNA level are delivered via an adenovirus or adeno associated virus via multiple doses.

In a further embodiment herein the delivery of said systems based on a DNA level is via a plasmid. In such plasmid compositions, the dosage should be a sufficient amount of plasmid to elicit a response. Plasmids of the invention will generally comprise (i) a promoter; (ii) a sequence encoding a Cas13 protein as defined herein, operably linked to said promoter; (iii) a selectable marker; (iv) an origin of replication; and (v) a transcription terminator downstream of and operably linked to (ii). The plasmid preferably also encodes the RNA components of a CRISPR complex such as comprising a nucleotide sequence encoding said gRNA as defined herein. In another further embodiment herein the delivery of said systems based on a DNA level is via a particle as defined elsewhere herein.

In some embodiments said systems based on RNA level are delivered in a liposome or in lipofectin formulations and the like and can be prepared by methods well known to those skilled in the art. Such methods are described, for example, in U.S. Pat. Nos. 5,593,972, 5,589,466, and 5,580,859, which are herein incorporated by reference. In other embodiments, said systems of the invention based on RNA level are delivered in a particle or a microvesicle. Thus, the present invention comprises besides plasmids or viral vectors as defined above liposomes, lipofectin formulations, particles or extracellular nanovesicles (such as exosomes) as delivery systems comprising said novel systems as defined herein.

Liposomes are spherical vesicle structures composed of a uni- or multilamellar lipid bilayer surrounding internal aqueous compartments and a relatively impermeable outer lipophilic phospholipid bilayer. Liposomes have gained considerable attention as drug delivery carriers because they are biocompatible, nontoxic, can deliver both hydrophilic and lipophilic drug molecules, protect their cargo from degradation by plasma enzymes, and transport their load across biological membranes and the blood brain barrier (BBB). Liposomes can be made from several different types of lipids; however, phospholipids are most commonly used to generate liposomes as drug carriers. Liposomal transfection reagents such as lipofectamine and other reagents on the market can effectively deliver RNA molecules.

In general, a particle is defined as a small object that behaves as a whole unit with respect to its transport and properties. Particles are further classified according to diameter. Coarse particles cover a range between 2,500 and 10,000 nm. Fine particles are sized between 100 and 2,500 nm. Ultrafine particles, or nanoparticles, are generally between 1 and 100 nm in size. The basis of the 100-nm limit is the fact that novel properties that differentiate particles from the bulk material typically develop at a critical length scale of under 100 nm. Particles delivery systems within the scope of the present invention may be provided in any form, including but not limited to solid, semi-solid, emulsion, or colloidal particles. The term “particles” also covers virus-like particles (VLPs) as known to a person skilled in the art. Thus, the present invention also comprises a VLP as RNA delivery system comprising the novel systems of the invention.

Exosomes are endogenous extracellular nanovesicles that transport RNAs and proteins, and which can deliver RNA to the brain and other target organs. Indeed, exosomes have been shown to be particularly useful in delivery siRNA, a system with some parallels to the RNA-targeting system. More details on the delivery systems with regard to DNA delivery (AAV, lentivirus etc.) or RNA delivery (liposome, particles, exosomes etc.) are described in US20200165594, which is herein incorporated by reference.

With regard to each system variant, the delivery of each variant can be different. When the system variant 1 (see FIG. 5) or 4 (see FIG. 8) may be delivered on DNA level or RNA level one delivery system as defined above may be used. When the system variant 2 (see FIG. 6) or 3 (see FIG. 7) may be delivered on DNA level or RNA level one delivery system as defined above may be used, but also two delivery systems of the same kind (e.g. two AAV vectors, two VLPs) as defined above can be used for said delivery. This may then refer to a separate delivery comprising two delivery systems as defined herein. In this context, the first construct of system variant 2 or 3 comprising at least one nucleotide sequence encoding said at least one Cas13 protein can be delivered prior to the second construct comprising at least one gRNA or at least one nucleotide sequence encoding said at least one gRNA to give time for CRISPR Cas13 protein to be expressed. The first construct might be administered 1-12 hours (preferably around 2-6 hours) prior to the administration of the second construct of said two variants. Alternatively, both constructs of system variant 2 or 3 can be administered together, which then refers to a joint delivery also comprising two delivery systems. Advantageously, a second booster dose of said second construct with regard to said gRNA can be administered 1-12 hours (preferably around 2-6 hours) after the initial administration of the first and the second construct of system variant 2 or 3.

### Composition

The present invention also relates to a composition comprising the novel systems as defined herein or the delivery systems as mentioned herein. According to the present invention, the composition as defined throughout the present invention may further comprise at least one carrier, diluent or excipient as defined elsewhere herein. Said terms can be used interchangeably. Preferably, the at least one carrier refers to at least one pharmaceutically acceptable carrier. Said pharmaceutically acceptable carrier (also called excipient or diluent) includes any excipient/carrier/diluent that does not itself elicit an adverse reaction harmful to the subject receiving the pharmaceutical composition. The present invention also relates to said composition as defined herein, which is a pharmaceutical composition. If the composition additionally comprises at least one pharmaceutically acceptable carrier, said composition refers to a pharmaceutical composition. Said pharmaceutical composition is thus used herein for therapeutic purposes. Moreover, the present invention relates to the use of said composition as disclosed herein above for the preparation of a pharmaceutical composition.

In accordance with the present invention, the term “pharmaceutical composition” relates to a composition for administration to a subject as defined herein, preferably a human. Pharmaceutical compositions or formulations are usually in such a form as to allow the biological activity of the active ingredient to be effective and may therefore be administered to a subject for therapeutic use as described herein. The pharmaceutical composition can be administered by inhalation, injection, infusion, or orally. Thus, the pharmaceutical composition may be a composition for oral, parenteral, trans-dermal, intra-luminal, intra-arterial, intra-venous, intra-thecal and/or intranasal administration or for direct injection into tissue. The pharmaceutical compositions can be administered to the subject at a suitable dose. The dosage regimen will be determined by the attending physician and by clinical factors. As is well known in the medical arts, dosages for any one patient depend upon many factors, including the patient's size, body surface area, age, the particular compound to be administered, sex, time and route of administration, general health, and other drugs being administered concurrently.

Suitable carriers are typically large, slowly metabolized macromolecules such as proteins, polysaccharides, polylactic acids, polyglycolic acids, polymeric amino acids, amino acid copolymers and lipid aggregates such as e.g. oil droplets or liposomes. The carrier used in combination with the (pharmaceutical) composition of the present invention may be water-based and forms an aqueous solution. An oil-based carrier solution containing the systems of the present invention is an alternative to the aqueous carrier solution. Either aqueous or oil-based solutions further contain thickening agents to provide the (pharmaceutical) composition with the viscosity of a liniment, cream, ointment, gel, or the like. Suitable thickening agents are well known to those skilled in the art.

Pharmaceutically acceptable carriers according to the present invention include, by the way of illustration and not limitation, disintegrants, binding agents, adhesives, wetting agents, polymers, lubricants, gliands, substances added to mask or counteract a disagreeable texture, taste or odor, flavors, dyes, fragrances, and substances added to improve appearance of the composition. Acceptable carriers include lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable carriers for soft gelatin capsules include vegetable oils, waxes, fats, semisolid and liquid polyols. Suitable carriers for the preparation of solutions and syrups include, without limitation, water, polyols, sucrose, invert sugar and glucose. Suitable carriers for injectable solutions include, without limitation, water, alcohols, polyols, glycerol, and vegetable oils. The pharmaceutical compositions can additionally include preservatives, solubilizers, stabilizers, wetting agents, emulsifiers, sweeteners, colorants, flavorings, buffers, coating agents, or antioxidants. Suitable pharmaceutical carriers are described in Remington's Pharmaceutical Sciences, Mack Publishing Company, a standard reference text in this field.

Further, the carriers of the (pharmaceutical) composition may also refer to diluents such as, e.g. water, saline, glycerol, ethanol, bacteriostatic water for injection (BWFI), Ringer's solution, dextrose solution, or aqueous solutions of salts and/or buffers etc. Furthermore, substances necessary for formulation purposes may be comprised in said compositions as acceptable carriers such as emulsifying agents, stabilizing agent, surfactants and/or pH buffering agents known to a person skilled in the art.

Said stabilizing agent/stabilizer may act as a tonicity modifier. The term “stabilizing agent” refers to an agent that improves or otherwise enhances stability of the formulation, in particular of the system comprised in said (pharmaceutical) composition. A stabilizing agent which is a tonicity modifier may be a non-reducing sugar, a sugar alcohol or a combination thereof. The tonicity modifiers of the (pharmaceutical) compositions of the present invention ensure that the tonicity, i.e. osmolarity, of the solution is essentially the same as normal physiological fluids and may thus prevent post-administration swelling or rapid absorption of the composition because of differential ion concentrations between the composition and physiological fluids. Preferably, the stabilizing agent/tonicity modifier is one or more of non-reducing sugars, such as sucrose or trehalose or one or more of sugar alcohols, such as mannitol or sorbitol, also combinations of non-reducing sugars and sugar alcohols are preferred.

Also, the pharmaceutical composition as defined herein may comprise one or more adjuvants. The term “adjuvant” is used according to its well-known meaning in connection with pharmaceutical compositions. Specifically, an adjuvant is an immunological agent that modifies, preferably enhances, the effect of such composition while having few, if any, desired immunogenic effects on the immune system when given per se. Suitable adjuvants can be inorganic adjuvants such as, e.g., aluminium salts (e.g., aluminium phosphate, aluminium hydroxide), monophosphoryl lipid A, or organic adjuvants such as squalene or oil-based adjuvants, as well as virosomes.

Said (pharmaceutical) composition of the present invention may be a liquid, preferably an aqueous, composition. Further comprised herein is a dried or frozen form of the (pharmaceutical) composition as defined herein. In this context, a frozen form may refer to a composition of at least about −20° C. Preferably, said (pharmaceutical) composition is a liquid, even more preferably an aqueous, composition. Thus, said (pharmaceutical) composition may be stored directly in liquid form for later use, stored in a frozen state and thawed prior to use, or prepared in dried form, such as a lyophilized, air-dried, or spray-dried form, for later reconstitution into a liquid form or other form prior to use. Thus, it is envisaged that a (pharmaceutical) composition described herein may be stored by any method known to one of skill in the art. Non-limiting examples include cooling, freezing, lyophilizing, and spray drying the formulation, wherein storage by cooling is preferred.

### In Vivo Therapeutic Applications

The present invention further refers to the delivery systems or the composition comprising the systems or the delivery systems as defined elsewhere herein for use as a medicament. Hence, the delivery systems or the composition comprising the systems or the delivery systems of the present invention can also be used for therapy, i.e. the treatment of a viral disease in a subject in need thereof as defined herein. Accordingly, the delivery systems or the composition comprising the systems or the delivery systems of the present invention is particularly suitable for use in a method of preventing or treating a viral disease in a subject in need thereof.

Hence, the present invention also provides for a method for the prevention or treatment of a viral disease in a subject, the method comprising administering a therapeutically effective amount of the delivery systems or the composition comprising the systems or the delivery systems of the present invention to a subject in need thereof.

As such the term “treat”, “treating” or “treatment” as used herein means to reduce (slow down (lessen)), stabilize or inhibit or at least partially alleviate or abrogate the progression of the symptoms associated with the respective disease. Thus, it includes the administration of the delivery systems or the composition comprising the systems or the delivery systems, preferably in the form of a medicament, to a subject, defined elsewhere herein. Those in need of treatment include those already suffering from the disease, here a viral disease as described elsewhere herein. Preferably, a treatment reduces (slows down (lessens)), stabilizes, or inhibits or at least partially alleviates or abrogates progression of a symptom that is associated with the presence and/or progression of a disease or pathological condition. “Treat”, “treating”, or “treatment” refers to a therapeutic treatment. In particular, in the context of the present invention, treating or treatment refers to an improvement of the symptom that is associated with said viral disease as defined elsewhere herein in a subject in need thereof. In this context, the term “treat”, “treating” or “treatment” refers to an antiviral therapy that directly attacks the viral target RNA molecules sequences of the specific viruses as defined herein. Thus the delivery systems or the composition comprising the systems or the delivery systems as defined herein may also be used as anti-viral therapeutic.

The term “prevent”, “preventing”, “prevention” as used herein refers to prophylactic or preventative measures, wherein the subject is to prevent an abnormal, including pathologic, condition in the organism which would then lead to the defined disease, namely said viral disease as defined herein. In other words, said terms refer to a medical procedure whose purpose is to prevent a disease meaning inhibiting that a subject will likely suffer from any future viral disease as defined herein. As used herein, such terms also refer to the reduction in the risk of acquiring or developing a given condition in a patient diagnosed with any viral disease as defined herein. Those in need of the prevention include those prone to having the disease, such as said viral disease as defined herein. In other words, those who are of a risk to develop such disease and will thus probably suffer from said disease in the near future. Thus, the delivery systems or the composition comprising the systems or the delivery systems as defined herein may also be used as a prophylaxis, not as a therapeutic agent for a subject as defined herein that has already been infected. This may prevent Cas13 protein from being overwhelmed by the viral RNA molecules.

The term “subject” when used herein includes mammalian and non-mammalian subjects. Preferably the subject of the present invention is a mammal, including human, domestic and farm animals, non-human primates, and any other animal that has mammary tissue. In some embodiment the mammal is a mouse. In some embodiment the mammal is a rat. In some embodiment the mammal is a guinea pig. In some embodiment the mammal is a rabbit. In some embodiment the mammal is a cat. In some embodiment the mammal is a dog. In some embodiment the mammal is a monkey. In some embodiment the mammal is a horse. In a most preferred embodiment the mammal of the present invention is a human. A subject also includes human and veterinary patients.

The uses, kits and compositions described in this document are generally applicable to both human and veterinary diseases. Where the subject is a living human who may receive treatment for a disease or condition as described herein, it is also addressed as a “patient”. In some embodiments the subject of the present invention is of a risk to develop said viral disease as described herein. In some embodiments the subject of the present invention suffers from said viral disease as described herein. The term “suffering” as used herein means that the subject is not any more a healthy subject. The term “healthy” means that the respective subject has no obvious or noticeable hallmarks or symptoms of the respective disease. This further means that the subject suffering from said viral disease is a subject “in need” of the respective treatment with the delivery systems or the composition comprising the systems or the delivery systems of the present invention.

The delivery systems per se or the composition comprising said systems or said delivery systems of the present invention for use in a method of preventing or treating a subject suffering from a viral disease as defined elsewhere herein are generally administered to the subject in a therapeutically effective amount. Said therapeutically effective amount is sufficient to inhibit or alleviate the symptoms of said viral disease. By “therapeutic effect” or “therapeutically effective” is meant that the conjugate for use will elicit the biological or medical response of a tissue, system, animal or human that is being sought by the researcher, veterinarian, medical doctor or other clinician. The term “therapeutically effective” further refers to the inhibition of factors causing or contributing to the disease or disorder. The term “therapeutically effective amount” includes that the amount of the agent when administered is sufficient to significantly improve the progression of the disease being treated or to prevent development of said disease. According to a preferred embodiment, the therapeutic effective amount is sufficient to alleviate or heal said viral disease as defined herein.

The therapeutically effective amount will vary depending on the delivery systems or the composition comprising the systems or the delivery systems of the present invention, the disease and its severity and on individual factors of the subject and/or also how the administration (also called the delivery) of said works. Therefore, the composition of the present invention will not in all cases turn out to be therapeutically effective, because the method disclosed herein cannot provide a 100% safe prediction whether or not a subject may be responsive to the detection system, since individual factors are involved as well. It is to expect that age, body weight, general health, sex, diet, drug interaction and the like may have a general influence as to whether or not the compound for use in the treatment of a subject suffering from said disease will be therapeutically effective.

The term “administering” or “administered” or “administration” used throughout various aspects of the present invention means that the delivery systems or the composition comprising the systems or the delivery systems as defined herein are given to the respective subject in an appropriate form and dose and using appropriate measures. The administration of the composition according to the present invention can be carried out by any method known in the art.

The administration of said delivery systems or the composition comprising the systems or the delivery systems in a therapeutically effective amount as defined elsewhere herein may be performed by inhalation, injection, infusion, or orally. The administration of said delivery systems or the composition comprising the systems or the delivery systems as defined elsewhere herein may be performed intraperitoneally, intravenously, intraarterially, subcutaneously, intramuscularly, parenterally, transdermally, intraluminally, intrathecally and/or intranasally or directly into tissue.

Where said delivery system per se or said composition as defined herein is to be administered by infusion, it can be dispensed with an infusion bottle containing sterile pharmaceutical grade water or saline. Where said delivery system per se or said composition as defined herein is administered by injection, an ampoule of sterile water for injection or saline can be provided so that the ingredients may be mixed prior to administration. Where said delivery system per se or said composition as defined herein is to be administered by inhalation, adequate inhalation devices may be used known to a person skilled.

In a particular embodiment, said administration of said delivery systems or said composition as defined herein may comprise the delivery to said subject via different methods based on whether said composition is delivered as a DNA or an RNA based system. If it is delivered as a DNA based system it may be administered using any vector as described herein such as adeno-associated virus (AAV) vector for AAV-mediated gene delivery or adenoviral or lentiviral vectors optimized for expression of said at least one gRNA and said at least one Cas13 gene and/or using particles such as nanoparticles or VLPs. If it is delivered as an RNA based system it may be administered using particular particles such as nanoparticles or VLPs and/or liposomes and/or exosomes comprising said delivery systems or said composition of the invention as it is known to a person skilled in the art, preferably via nanoparticles or VLPs.

The viral disease, which is prevented or treated by the use of said delivery systems or said composition as defined herein may refer to a disease caused by an RNA virus or a retrovirus as defined elsewhere herein, preferably caused by an RNA virus. In specific embodiments. the viral infection is caused by a double-stranded RNA virus, a positive sense RNA virus, a negative sense RNA virus or a combination thereof. In a more preferred embodiment, the viral disease, which is prevented or treated by the use of said delivery systems or said composition as defined herein is any one of a coronavirus disease, influenza A, ebola, measles, hepatitis C, tick-borne encephalitis (TBE). Venezuelan Equine Encephalitis (VEE) viral infection, dengue fever, yellow fever, bunya virus disease, respiratory syncytial virus (RSV) disease or zika fever, even more preferably coronavirus or influenza disease, most preferably coronavirus. The “coronavirus” may be SARS-CoV, SARS-CoV2 or MERS or a related new zoonotic or mutant coronavirus. The Coronavirus group consists of enveloped positive stranded RNA viruses belonging to the family Coronaviridae and comprise subtypes referred to as Alpha-, Beta-, Gamma- and Delta coronavirus. Alpha and Beta affect mammals, while Gamma affects birds and Delta can affect both. The coronavirus family comprises several well-known disease-causing members. The Betacoronavirus family has so far posed the biggest risk to humans and now includes the most well-known virus targets including Severe Acute Respiratory Syndrome coronavirus (SARS-CoV-1), Middle East Respiratory Syndrome coronavirus (MERS-CoV), and the newest form to emerge, the novel coronavirus (SARS-CoV-2). In a most preferred embodiment, said viral disease, which is prevented or treated by the use of said delivery systems or said composition as defined herein, is the COVID-19 disease. In the case of COVID-19, the clinical spectrum of SARS-CoV-2 infection appears to be wide, encompassing asymptomatic infection, mild upper respiratory tract illness, and severe viral pneumonia with respiratory failure and even death, with many patients being hospitalized.

In alternative embodiments, the composition for the use in the treatment of said viral disease of the present invention may also be administered in combination with an additional therapeutic agent (drug). Drugs or therapeutic agents useful in this regard include without limitation drug-like molecules, proteins, peptides, and small molecules. Protein therapeutic agents include, without limitation peptides, enzymes, antibodies, structural proteins, receptors and other cellular or circulating proteins as well as fragments and derivatives thereof, preferably an additional therapeutic agent/drug in the context of the present invention may be a drug for the use in viral diseases as described elsewhere herein, especially for combinatorial therapy in said diseases. Said combination according to the present invention can be administered as a combined formulation or separate from each other.

Also comprised by the present invention is a method of preventing or treating a viral disease in a subject, the method comprising administering to the subject a therapeutically effective amount of said delivery systems or said composition as defined herein. Also comprised herein is the use of said delivery systems or said composition as defined herein for the manufacture of a medicament for therapeutic application in a viral disease in a subject. The definitions and embodiments made with regard to the first and second medical uses may be applied in this context as well.

### Kit

The present invention also relates to a kit comprising the systems as defined herein by the present invention. Thus, when a kit comprises the systems of the present invention, said systems may be provided in a vial or a container, preferably also comprising in said vial or container at least one carrier as defined herein. Further, said kits may be associated with a notice in the form prescribed by a governmental agency regulating the manufacture, use or sale of pharmaceuticals or biological products, reflecting approval by the agency of the manufacture, use or sale of the product for human administration or diagnostics. Said kits may comprise the systems as defined herein, preferably in a vial or container, in dried form, such as a lyophilized, air-dried, or spray-dried form (in form of a powder), for later reconstitution into a liquid form or other form prior to use. Further, said kits may also comprise the systems, preferably in a vial or container, in a frozen state, being thawed prior to use.

The kits according to the present invention may also comprise a delivery system comprising said systems as defined herein such as a vector (e.g., AAV, adenovirus, lentivirus, plasmid) and/or a particle for a DNA delivery of said systems. Also comprised by the present invention may be a kit comprising a delivery system comprising said systems as defined herein such as particles and/or liposomes and/or exosomes for a RNA delivery of said systems. Such delivery systems may also be comprised in the one or more vials or containers of the kits as defined above or in additional one or more vials or containers of said kit, preferably further comprising in said one or more vials or containers any excipient suitable for said delivery system to be mixed with/contacted with.

Additionally or alternatively, the kit according to the present invention may also comprise a label. A label as used herein may refer to a compound capable of targeting said systems. Said compound may refer to an antibody, beads coupled/coated to an antibody f.e. dynabeads coupled/coated to an antibody, preferably which is used for classical purification processes such as chromatography when the composition may be produced on the protein level. Further, said label can be oligodT magnetic beads. Such beads may be capable of binding to the polyA tail of the full-length RNA, when the composition on the RNA level is produced and then further purified as known to the person skilled in the art. In some embodiments, said label may also be comprised in the one or more containers or vials of the kit as defined above comprising said systems or in additional one or more vials or containers of said kit, preferably further comprising in said one or more vials or containers any excipient suitable for said label to be mixed with/contacted with.

Said kit as defined herein may also comprise a manual comprising instructions for applying the comprised systems (additionally also comprising the delivery system and/or the label) as defined herein to prevent or treat a viral disease in a subject as defined also herein.

### In Vitro Applications

The present invention also relates to a method of producing the systems of the present invention. In this context, the systems are produced starting from the synthesizing of said at least one nucleotide sequence encoding said at least one Cas13 protein and said at least one gRNA or said nucleotide sequence encoding said at least one gRNA as defined herein as well as of said at least one viral 5′ UTR or said at least one nucleotide sequence encoding said at least one 5′ UTR and/or said at least one viral 3′ UTR or said at least one nucleotide sequence encoding said at least one 3′ UTR by means of genetic engineering methods. Thus, the end product of the provided production method is the CRISPR system of the present invention, either in the form of a DNA based system or an RNA based system.

Thus, when the term “synthesizing by means of genetic engineering” is used herein with regard to the DNA based system, it refers to the in vitro usage of artificial gene synthesis (for said DNA sequence encoding said Cas13 protein, said DNA sequence encoding said gRNA and DNA sequence encoding UTR(s)). Such synthesis refers to a method that is used in synthetic biology to construct and assemble genes from nucleotides de novo as it is known to a person skilled in the art. When the term “synthesizing by means of genetic engineering” is used herein with regard to the RNA based system, it refers to the in vitro usage of RNA synthesis (Cas13 mRNA, gRNA and UTR(s) mRNA as defined herein) as known to a person skilled in the art. After the synthesizing as defined above, the method may further comprise the obtaining of the produced systems.

When the systems are produced on the DNA level (DNA sequence encoding said Cas13 protein, DNA sequence encoding said gRNA and DNA sequence encoding UTR(s)), the term “obtain” or “obtaining” when used in this respect can be understood in that the produced systems are extracted from the used (reaction) buffer after the synthesizing, by using f.e. artificial gene synthesis, and potentially purified before said systems can be used. Such classical purification process of a DNA based system may comprise the usage of silica columns as known to the person skilled in the art. When the systems are produced on the RNA level (Cas13 mRNA, gRNA and UTR mRNA), the term “obtain” or “obtaining” when used in this respect can be understood in that the produced systems are extracted from the used (reaction) buffer after the nucleic acid coding, by using f.e. RNA synthesis, and potentially purified before said systems can be used. Such classical purification process of an RNA based system may comprise the usage of oligodT magnetic beads as known to the person skilled in the art. Such beads are capable of binding to the polyA tail of the RNAs of the components of said CRISPR system, wherein by-products may be separated, thus achieving better purity of said components The term “recover” or “recovering” can be used interchangeably with the term “obtain” or “obtaining” herein.

### EXAMPLES OF THE INVENTION

### Materials and Methods

### Cloning of Key Constructs.

Cas13 coding sequences, viral UTR/packaging sequences and additional elements were ordered as synthetic fragment (gBlock, IDT or Twist Fragment, Twist Bioscience) and cloned into a pCAG backbone.

### Standard RNA Preparation.

Templates for in vitro transcription were generated by linearization of plasmid templates. Digests were purified (Monarch DNA Cleanup Kit, New England Biolabs), used for mRNA synthesis (HiScribe T7 Kit, New England Biolabs), capped (Vaccina Capping System, New England Biolabs) and polyadenylated (PolyA Polymerase, New Englang Biolabs). Synthesized RNA was purified (Monarch RNA Cleanup, New England Biolabs) and stored at −80° C.

### Cell Lines and Cultivation.

All experiments were performed in HEK293T cells. Cells were maintained at 37° C. in 5.0% CO₂, H₂O saturated atmosphere. DMEM medium with 10% FBS was used for maintenance.

### Cas13 Co-Replication and SARS-CoV-2 Inhibition.

HEK293-ACE2 cells were transfected with different co-replication RNA constructs by using JetMessenger reagent. 24 h after the transfection cells were infected with SARS-CoV-2-GFP under BSL3 conditions. Viral replication was monitored in an Incucyte S3 live cell imaging device in green (virus replication) and red (Cas13 replication) channel.

### Knockdown Efficiencies Measured via Luciferase Assays.

Cells were transfected with different Cas13 systems together with either mRNA or DNA coding for Nanoluciferase. 24-72 h after the transfection cells were lysed and Nanoluciferase was measured (NanoGlo Assay, Promega). In experiments where the on-target and off-target activity was quantified, Nanoluciferase along with Firefly Luciferase was transfected in the same well. 24-72 h after the transfection cells were lysed and both luciferases were measured independently (NanoGlo Dual Luciferase Assay, Promega).

### Assessment of Cas13 Protein Stability.

72 h after the transfection with different Cas13 variants, cells were lysed in M-PER buffer (Thermo Fisher) supplemented with Halt Protease Inhibitor Cocktail (Thermo Fisher), Lysates were prepared in Laemmli Buffer (Sigma-Aldrich) and run on a SDS PAGE (Thermo Fisher). Proteins were transferred to a PVDF membrane overnight at 20V, blocked, incubated with primary M2 Anti-FLAG (Merck Millipore) antibody to stain for Cas13 and imaged with Amersham ECL Prime substrate (Sigma-Aldrich).

### Cas13 Protein Localization.

72 h after the transfection with different Cas13 variants cells were fixed in formalin, permeabilized and incubated with primary M2 Anti-FLAG (Merck Millipore) antibody. After incubation with secondary antibody cells were additionally DAPI stained and imaged and quantified at Cellinsight CX7 High Content Imaging System (Thermo Fisher).

### Results

### Example 1: Implementation of Co-Replication and -Distribution

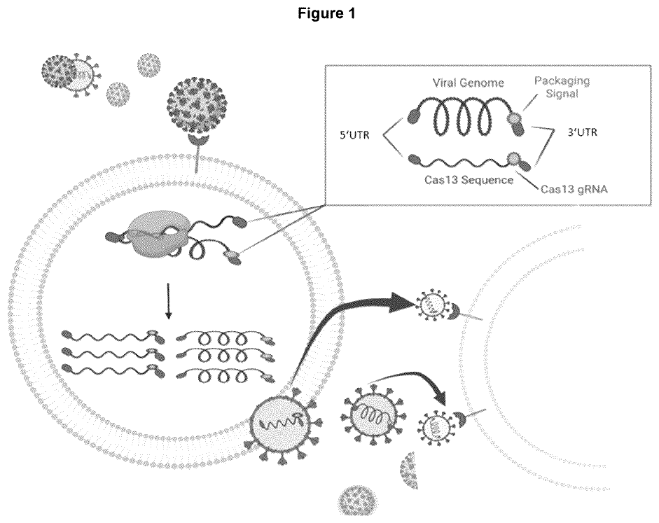

RNA viruses replicate their genomes via an RNA-dependent RNA polymerase. The polymerase recognizes structural elements of the RNA genome to initiate replication. To leverage the viral replication mechanism for an adaptive Cas13-based therapy, the Cas13 coding sequence was flanked by viral replication elements to induce co-replication of Cas13 in trans along with the viral genome (FIG. 1). To implement this approach for the bipartite Cas13 system (coding sequence and gRNA) four constructs were generated: 1) the gRNA is placed before the 3′UTR of the Cas13 coding sequence. Both components a present on a single molecule and flanked by viral replication recognition sequences, inducing trans-replication (FIG. 5A). 2) The gRNA and Cas13 coding sequence are present on two separate molecules. Both molecules are flanked by the viral replication recognition sequences, to induce trans-replication (FIG. 6A). 3) Only the Cas13 coding sequence is flanked by viral replication recognition sequences and therefore trans-replication competent. The gRNA is not modified and therefore replication incompetent (FIG. 7A). 4) Both structural elements are present on a single RNA molecule flanked by viral replication recognition signals and therefore replicated. The gRNA is split into two halves and reconstituted during the discontinuous replication process (FIG. 8A).

Besides the replication element, the viral genome encodes a packaging signal which is recognized by the viral capsid proteins. This packaging signal is integrated into the previous constructs 1-4 to induce not only co-replication but also co-distribution by packaging the modified Cas13 into virus-derived particles (FIG. 5B, FIG. 6B, FIG. 7B, FIG. 8B).

### Example 2: Modelling of the Antiviral Effect of Co-Replication/-Distribution

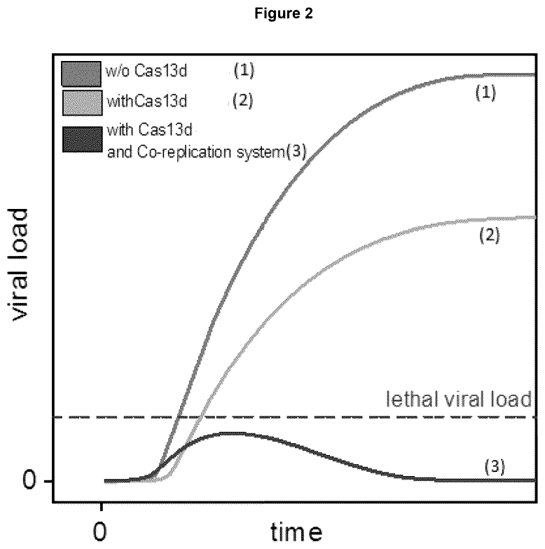

A non-replication competent Cas13 system inhibits the viral replication to a certain degree but might not be able to cope with the highly potent viral replication machinery. Therefore, a co-replication and co-distribution system was established to directly link the viral load to the antiviral treatment. A high viral load will induce strong replication of the modified Cas13 RNA, upon full clearance of the virus, the Cas13 construct is not replication-competent anymore and therefore degraded in a short period. Mathematical modeling of this system suggests a strong inhibitory effect of such a co-replication and co-distribution system, leading to full viral clearance (FIG. 2).

### Example 3: Modelling of the Antiviral Effect of Co-Replication/-Distribution

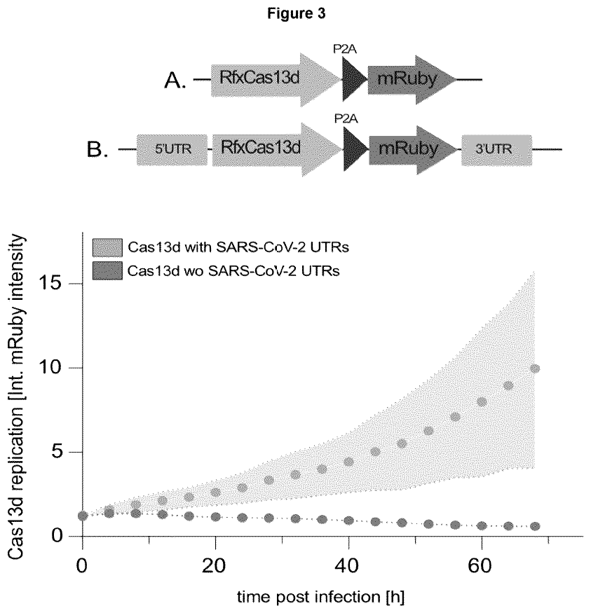

An mRuby3 reporter expressing Cas13 construct, flanked by the SARS-CoV-2 UTRs which are recognized by the viral polymerase, was generated to monitor the co-replication rate of the construct related to the SARS-CoV-2 viral load. A non-replication competent version of the construct constantly degraded over time, while the RNA levels of the co-replication competent construct increased over time (FIG. 3).

### Example 4: Antiviral Effect of the Co-Replication/-Distribution System

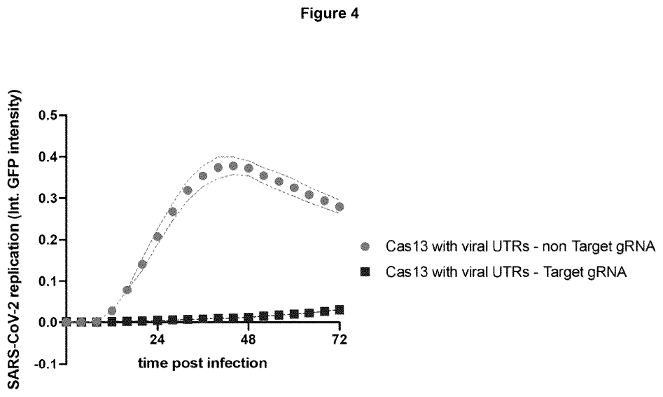

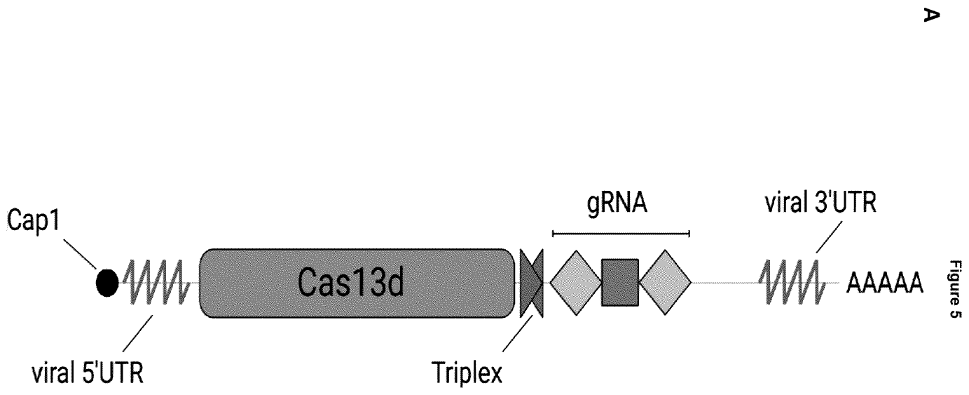

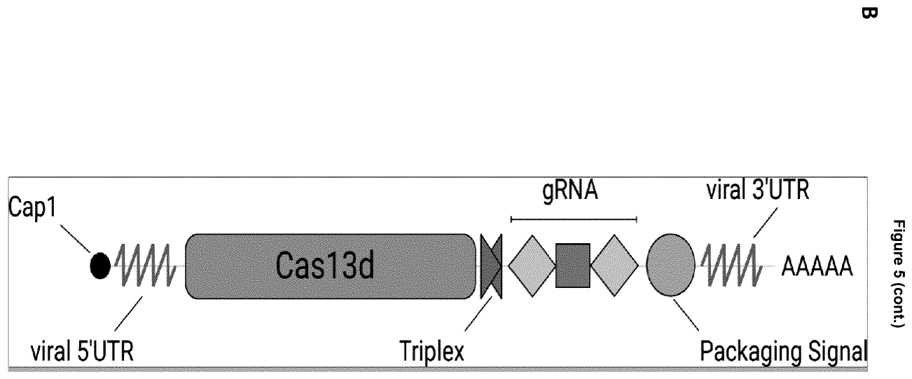

The antiviral effect of the co-replication Cas13 construct was measured by co-transfecting the RNA along with a gRNA targeting the SARS-CoV-2 genome. A non-target gRNA control did not impact the viral load, but in the target gRNA condition, the viral replication was strongly inhibited (FIG. 4).

### Example 5: Characterization of Optimal Knockdown Conditions for Cas13

To find the optimal composition of a Cas13-based knockdown tool, Cas13d variants and miRNA-based RNA interference were directly compared in a Firefly Luciferase knockdown assay. The known Cas13d-NLS with 22 bp gRNA yield a moderate knockdown efficiency, which was improved by increasing the gRNA length to 30 bp and by fusing the Cas13 protein to a NLS and NES together. This optimized system is even more efficient than latest generation RNA interference (FIG. 9).

Originally, the optimal gRNA length for Cas13d was found to be 22 bp in an in vitro assay with purified components (Konermann et al. (2018). Cell 173(3): 665-676). The inventors characterized the knockdown efficiency of different gRNAs lengths in a mammalian cell line, by targeting a co-transfected Nanoluciferase. In contrast to previous in vitro work the optimal gRNA length was in the range of 26 to 30 bp (FIG. 10).

### Example 6: Exploitation of Different Strategies to Transfer Cas13d to the Cytosol

The inventors compared the knockdown efficiency of Cas13d proteins with different localization signals in a luciferase-based assay. Pol III driven gRNAs are expressed and remain in the nucleus, therefore Cas13d exported to the cytosol (NES) is hardly active. Additionally, Western Blot analysis showed that the protein is unstable if it is not bound to a gRNA in the same compartment. A Cas13d variant which is imported to the nucleus (NLS) is active and stable because of being present in the same compartment as the gRNA and forming a gRNA/Cas13d complex. For a Cas13d fusion protein consisting of both localization signals (NLS-NES) a shuffling mechanism was expected. The protein is imported to the nucleus (NLS), picks up the gRNA and then being exported to the cytosol (NES) again, where the majority of the luciferase mRNA is targeted for degradation. This NLS-NES strategy stabilized the protein in the cytosol and maximized the knockdown efficiency. A complementary strategy in which the gRNA itself is exported to the cytosol by expression from a Pol Il promoter supports the hypothesis, because in this case, Cas13d-NES is most active. By directly comparing the two strategies, Cas13d-NLS-NES is most efficient (FIG. 11).

### Example 7: Illustration of Cas 13d Variants Fused to Different Localization Signals

Cas13d was fused to different single or tandem localization signals to induce a nuclear/cytosolic shuttling. A co-transfected nanoluciferase was targeted by two gRNAs in combination with different localized Cas13 variants. A combination of two NLS, along with one NES signal (Cas13d-NLS-NES) was found to be optimal, likely due to sufficient nuclear import to pick up the gRNA and cytosolic localization to efficiently target the mainly cytosolic nanoluciferase mRNA (FIG. 12).

### Example 8: Localization of Cas13d Protein Variants Fused to Different Localization Signals

To analyze the impact of different single or tandem localization sequences on the localization of Cas13d immunohistochemistry was performed. Different fusion constructs were transfected and imaged in a high-content analysis system (FIG. 13A). Subsequently, fluorescence intensity measurements for cytosolic and nuclear fractions were performed for 100 cells for each construct. Analysis of cytosolic to nuclear distribution revealed a gradual shift of the protein localization to the cytosol for decreasing NLS and increasing NES signals (FIG. 13B). Cas13d-NES-NLS shows an intermediate localization, supporting the hypothesized shuttling mechanism.

### Example 9: Comparison of Knockdown Efficiencies for Two gRNAs Targeting a Co-Transfected Nanoluciferase for NES or Tandem NLS-NES Fused to Cas13

Cas13d-NLS-NES travels to the nucleus to pick up the gRNA while CTE fused gRNAs are exported to the cytosol by itself. The combination of both export systems could have an additive effect. Therefore, HEK293T cells were transfected with two gRNAs targeting nanoluciferase, either with or without fused CTE motif and either Cas13-NES or Cas13-NLS-NES. 48 h after the transfection, knockdown efficiencies were measured. Both gRNA export strategies alone performed similar, but the synergistic effect of both export systems further maximized the efficiency (FIG. 14).

### Example 10: Illustration of Different crRNA Export Strategies

Conventual gRNAs are expressed by polymerase III promoters (e.g. U6 promoter) and therefore remain in the nucleus of expressing cells. To target cytoplasmic RNAs, different gRNA export strategies were exploited. Polymerase II driven gRNAs (e.g. CAG promoter) are capped, polyadenylated and therefore exported. Alternatively, viral RNA export elements, such as CTE or VARdm drive the export of gRNAs from the nucleus to the cytosol (FIG. 15A). These export strategies were applied to two gRNAs, targeting a co-transfected nanoluciferase and compared for either a cytosolic or nuclear Cas13 protein (FIG. 15B). As previously shown, conventual pol III driven gRNAs are more efficient in combination with a nuclear Cas13 system. Pol II gRNA expression and fusing viral export motifs favor a cytosolic over a nuclear Cas13 protein. gRNAs fused to the viral CTE motif are most efficient in driving cytosolic knockdown by Cas13d-NES.

1. A clustered, regularly interspaced, short palindromic repeats (CRISPR) system comprising i) at least one nucleotide sequence encoding at least one CRISPR-associated protein 13 (Cas13); and ii) at least one guide RNA (gRNA) or at least one nucleotide sequence encoding said at least one gRNA capable of hybridizing with one or more viral target RNA molecules, wherein said system comprises a viral 5′ untranslated region (UTR) or a nucleotide sequence encoding said 5′ UTR and/or a viral 3′ UTR or a nucleotide sequence encoding said viral 3′ UTR, wherein a viral replicase recognition sequence is comprised in at least any one of said 5′ UTR or in the nucleotide sequence encoding said 5′ UTR, or said 3′ UTR or in the nucleotide sequence encoding said 3′ UTR, and wherein said system does not comprise a nucleotide sequence encoding a viral replicase and wherein said viral replicase recognition sequence is from the same RNA virus as the one or more viral target RNA molecules.

2. The system of claim 1, wherein said at least one nucleotide sequence encoding said at least one Cas13 protein and said at least one gRNA or said at least one nucleotide sequence encoding said at least one gRNA are on the same nucleotide construct of said system, the construct which further comprises said viral 5′ UTR or said nucleotide sequence encoding said 5′ UTR and/or said viral 3′ UTR or said nucleotide sequence encoding said viral 3′ UTR, wherein said viral replicase recognition sequence is comprised in at least any one of said 5′ UTR or in the nucleotide sequence encoding said 5′ UTR, or said 3′ UTR or in the nucleotide sequence encoding said 3′ UTR.

3. The system of claim 2, wherein said at least one gRNA or said nucleotide sequence encoding said gRNA is split in two parts and wherein
i) one part is integrated at the 5′ end of said at least one nucleotide sequence encoding said at least one Cas13 protein; and
ii) the other part of said gRNA or said nucleotide sequence encoding said gRNA is integrated at the 3′ end of said at least one nucleotide sequence encoding said at least one Cas13 protein.

4. The system of claim 1, wherein said at least one nucleotide sequence encoding said at least one Cas13 protein and said at least one gRNA or said at least one nucleotide sequence encoding said at least one gRNA are on two different nucleotide constructs of said system, the first construct comprising said at least one nucleotide sequence encoding said at least one Cas13 protein further comprises said viral 5′ UTR or said nucleotide sequence encoding said 5′ UTR and/or said viral 3′ UTR or said nucleotide sequence encoding said viral 3′ UTR, wherein said viral replicase recognition sequence is comprised in at least any one of said 5′ UTR or in the nucleotide sequence encoding said 5′ UTR, or said 3′ UTR or in the nucleotide sequence encoding said 3′ UTR; and
the second construct comprising said at least one gRNA or said at least one nucleotide sequence encoding said at least one gRNA.

5. The system of claim 4, wherein the second construct comprising said at least one gRNA or said at least one nucleotide sequence encoding said at least one gRNA also comprises said viral 5′ UTR or said nucleotide sequence encoding said 5′ UTR and/or said viral 3′ UTR or said nucleotide sequence encoding said viral 3′ UTR, wherein said viral replicase recognition sequence is comprised in at least any one of said 5′ UTR or in the nucleotide sequence encoding said 5′ UTR, or said 3′ UTR or in the nucleotide sequence encoding said 3′ UTR.

6. The system of any one of the preceding claims, wherein said system further comprises at least one nucleotide sequence encoding at least one viral packaging signal which is comprised by said nucleotide construct of said system comprising said at least one nucleotide sequence encoding said at least one Cas13 protein.

7. The system of claim 6, wherein said at least one nucleotide sequence encoding said at least one viral packaging signal is also comprised by said construct comprising said at least one gRNA or said at least one nucleotide sequence encoding said at least one gRNA as defined by claim 5.

8. The system of any one of the preceding claims, wherein said Cas13 protein is a Cas13d protein.

9. The system of claim 8, wherein said Cas13d protein is derived from the genus of Ruminococcus, preferably from Ruminococcus flavevaciens.

10. The system of any one of the preceding claims, wherein said at least one nucleotide sequence encoding said at least one Cas13 protein
i) is fused with at least one nucleotide sequence encoding at least one nuclear localization signal (NLS) fused to at least one nucleotide sequence encoding at least one nuclear export sequence (NES), or
ii) is fused with at least one nucleotide sequence encoding at least one NLS or with at least one nucleotide sequence encoding at least one NES and wherein said at least one nucleotide sequence encoding said at least one gRNA is fused with at least one nucleotide sequence encoding at least one viral export element.

11. The system of claim 10, wherein said at least one nucleotide sequence encoding said at least one Cas13 protein
i) is fused with at least one nucleotide sequence encoding said at least one NLS fused to at least one nucleotide sequence encoding said at least one NES, or
ii) is fused with at least one nucleotide sequence encoding said at least one NLS or with at least one nucleotide sequence encoding said at least one NES,
via a nucleotide sequence encoding a peptide linker.

12. The system of any one of claims 10 i)-11 i), wherein said at least one nucleotide sequence encoding said at least one Cas13 protein is fused with two nucleotide sequences both encoding a NLS fused to one nucleotide sequence encoding a NES.

13. The system of claim 12, wherein the two NLS encoded by said two nucleotide sequences comprise the SV40 NLS having the amino acid sequence as depicted in SEQ ID NO: 5 and the NLS consensus sequence having the amino acid sequence as depicted in SEQ ID NO: 3 and the one NES encoded by said one nucleotide sequence comprises the HIV NES having the amino acid sequence as depicted in SEQ ID NO: 4.

14. The system of any one of the preceding claims, wherein said gRNA has a length of at least about 23 nucleotides.

15. The system of any one of the preceding claims, wherein said gRNA has a length of between about 26 to about 30 nucleotides.

16. The system of any one of the preceding claims, wherein said gRNA has at least about 68% complementary sequence identity to said one or more viral target RNA molecules.

17. The system of any one of the preceding claims, wherein said gRNA is capable of hybridizing to (a) 5′- and/or 3′-untranslated region(s) of said one or more viral target RNA molecules.

18. The system of any one of the preceding claims, wherein said nucleotide sequence encoding said gRNA is fused with a tRNA or a ribozyme.

19. The system of claim 4, wherein said gRNA of said second construct comprises the replacement of the 5′ and/or 3′ terminal nucleotides by 2′-O-methyl-3′P-thioate.

20. The system of any one of claims 1-9, 10i), and/or any one of claims 11-19, wherein said nucleotide sequence encoding said gRNA is fused with at least one nucleotide sequence encoding at least one viral export element.

21. The system of claim 10ii) or 20, wherein said at least one viral export element is a constitutive transport element (CTE) or adenovirus VA1 RNA (VARdm).

22. The system of any one of the preceding claims, wherein said system inactivates viral ssRNA.

23. A delivery system comprising the system of any one of the preceding claims.

24. A composition comprising the system of any one of claims 1-22 or the delivery system of claim 23.

25. The composition of claim 24, further comprising at least one pharmaceutically acceptable carrier.

26. The composition of claim 24 or 25 for use in therapy.

27. The composition of claim 24 or 25 for use in a method of preventing or treating a viral disease in a subject, the method comprising administering to the subject a therapeutically effective amount of the composition of claim 24 or 25.

28. The composition for the use of claim 27, wherein the viral disease is caused by a RNA virus.

29. The composition for the use of claim 28, wherein the viral disease is any one of a coronavirus disease, influenza A, ebola, measles, hepatitis C, tick-borne encephalitis (TBE), Venezuelan Equine Encephalitis (VEE) viral infection, dengue fever, yellow fever, bunya virus disease, respiratory syncytial virus (RSV) disease or or zika fever.

30. The composition for the use of claim 29, wherein the viral disease is the COVID-19 disease.

31. A kit comprising the system of any one of claims 1-22.

32. The kit of claim 31, further comprising a delivery system and/or a label.

33. A method of producing the system of any one of claims 1-22, comprising
a) synthesizing said at least one nucleotide sequence encoding said at least one Cas13 protein, said at least one gRNA or said at least one nucleotide sequence encoding said at least one gRNA and said viral 5′ UTR or said nucleotide sequence encoding said 5′ UTR and/or said viral 3′ UTR or said nucleotide sequence encoding said 3′ UTR as defined in any one of claims 1-22 by means of genetic engineering methods, thereby producing said system; optionally
b) obtaining said produced system of step a).


SEQUENCE LISTING (10 sequences)

SEQ ID NO: 1 - protein, 967 aa, Rumniococcus flavefaciens
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 2 - protein, 7 aa, synthetic construct
  REGION 1..7: note = SV40 NLS
PKKKRKV

SEQ ID NO: 3 - protein, 8 aa, synthetic construct
  REGION 1..8: note = NLS consensus sequence
PAAKKKLD

SEQ ID NO: 4 - protein, 11 aa, synthetic construct
  REGION 1..11: note = HIV NES
LQLPPLERLTL

SEQ ID NO: 5 - protein, 10 aa, synthetic construct
  REGION 1..10: note = SV40 NLS extended
PPKKKRKVED

SEQ ID NO: 6 - protein, 9 aa, synthetic construct
  REGION 1..9: note = c-myc NLS
PAAKRVKLD

SEQ ID NO: 7 - protein, 16 aa, synthetic construct
  REGION 1..16: note = nucleoplasmin NLS
KRPAATKKAGQAKKKK

SEQ ID NO: 8 - DNA, 72 bp, synthetic construct
  misc_feature 1..72: note = Murid gammaherpesvirus 68
  source 1..72: mol_type = other DNA
gccagggtagctcaattggtagagcatcaggctagtatcctgtcggttccggttcaagtccgggccctggtt

SEQ ID NO: 9 - DNA, 84 bp, synthetic construct
  misc_feature 1..84: note = Hepatitis delta virus
  source 1..84: mol_type = other DNA
ggccggcatggtcccagcctcctcgctggcgccggctgggcaacattccgaggggaccgtcccctcggtaatggcgaatgggac

SEQ ID NO: 10 - RNA, 28 nt, synthetic construct
  misc_feature 1..28: note = gRNA of SARS-CoV-2
  source 1..28: mol_type = other RNA
ttaaaatcacatggggatagcactacta